(19) United States
(12) Patent Application Publication
(10) Pub. No.: US 2006/0052324 A1
(43) Pub. Date: Mar. 9, 2006

(54) Methods and compositions for cell activation

(76) Inventors: Steven E. Artandi, Stanford, CA (US); Kavita Y. Sarin, Mountain View, CA (US); Maja K. Artandi, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/198,908

(22) Filed: Aug. 4, 2005

Related U.S. Application Data
(60) Provisional application No. 60/599,604, filed on Aug. 5, 2004.

(51) Int. Cl. A61K 48/00; C12N 15/87

(52) U.S. Cl. 514/44; 435/455

(57) ABSTRACT

Methods and compositions for activating a cell are provided. In practicing the subject methods, a cell including a coding sequence for either a telomerase reverse transcriptase (TERT) or a telomerase RNA component (TERC) is activated by conditionally increasing expression of the coding sequence. Also provided are transgenic animals and systems for practicing the subject methods.

### CROSS-REFERENCE

This application claims the benefit of U.S. Provisional Application No. 60/599,604, filed Aug. 5, 2004, which application is incorporated herein by reference in its entirety.

### GOVERNMENT RIGHTS

This invention was made with government support under federal grant no. 5KO8 CA082176-04 awarded by the National Cancer Institute of the National Institutes of Health. The United States Government may have certain rights in this invention.

### BACKGROUND OF THE INVENTION

Telomeres, which define the ends of chromosomes, consist of short, tandemly repeated DNA sequences loosely conserved in eukaryotes. Human telomeres consist of many kilobases of (TTAGGG)_{N} together with various associated proteins. Small amounts of these terminal sequences or telomeric DNA are lost from the tips of the chromosomes during the S phase of the cell cycle because of incomplete DNA replication. Many human cells progressively lose terminal sequence with cell division, a loss that correlates with the apparent absence of telomerase in these cells. The resulting telomeric shortening has been demonstrated to limit cellular lifespan, thereby resulting in cellular senescence and inactivation.

Telomerase is a ribonucleoprotein (RNP) that uses a portion of its RNA moiety as a template for telomeric DNA synthesis. The catalytic core of telomerase is comprised of two essential components: TERT, the telomerase reverse transcriptase, and TERC, the telomerase RNA component. Telomerase synthesizes telomeres through reverse transcription of the template sequence encoded in TERC and through protein interactions that facilitate telomere engagement. Genetic studies in yeast, murine, and human cells have established that TERT and TERC are obligate partners in telomere synthesis; inactivation of either subunit abrogates enzymatic activity and prevents telomere addition, leading to progressive telomere shortening as a consequence of the end replication problem. Telomere shortening ultimately leads to telomere uncapping, a change in telomere structure associated with loss of end protection that results in both checkpoint activation and chromosomal end-to-end fusion.

According to this well-validated paradigm, telomerase functions primarily to prevent telomere uncapping through enzymatic extension of telomeres. Telomerase is thought to serve a similar function during tumor development where it prevents telomere shortening and uncapping, thus enabling cancer cells to proliferate in an unlimited fashion.

A general need exists for the regulation and control of cell cycle stages, e.g., control of progression of a cell from a quiescent state to an active state, control of progression of a cell from a non-proliferating state to a proliferating state, and the like. Regulation and control of cell cycle stage, e.g., from a quiescent state to an active state, is beneficial for a number of diseases or disorders related to cell proliferative capacity and senescence, wherein the disorder results from the cells entering a quiescent state (i.e., loss of proliferative capacity), and where activation (i.e., a proliferative state) will contribute to treatment of the disorder. Accordingly, there continues to be a need for development of such methods.

Relevant Literature

U.S. patents of interest include: U.S. Pat. Nos. 6,166,178; 6,337,200; and 6,309,867. Also of interest are: Cheong et al., 2003, Exp. Mol. Med., 35(3):141-153; Gonzalez-Suarez et al., 2001, EMBO J., 20(11): 2619-2630; Ramirez et al., 1997, J. Invest. Dermatol., 108(1):113-117; Harle-Bachor et al., 1996, PNAS, 93(13):6476-6481; and Rochet et al., 1994, Cell, 76(6):1063-1073.

### SUMMARY OF THE INVENTION

Methods and compositions for cell activation are provided. In practicing the subject methods, cell activation is achieved by conditionally increasing expression of either a telomerase reverse transcriptase (TERT) or a telomerase RNA component (TERC). Also provided are transgenic animals and systems for practicing the subject methods.

### FEATURES OF THE INVENTION

A feature of the present invention provides a method for activating a cell by conditionally increasing transcription of a coding sequence of either (e.g., only one of) a telomerase reverse transcriptase (TERT), or a telomerase RNA component (TERC) in the cell in a manner sufficient to activate the cell. In some embodiments, the subject method conditionally increases transcription of a TERT coding sequence. In other embodiments, the subject method conditionally increases transcription of a TERC coding sequence. Such a cell includes a hair follicle cell; a pancreatic islet cell; a neuronal cell; a bone marrow cell; and the like. Such a cell also includes a stem cell or progenitor cell in the hair follicle, bone marrow, pancreas, central nervous system, bone and cartilage, liver, and the like. The methods may be performed in vitro or in vivo. In some embodiments, the cell is present in a mammal, such as a human.

In some embodiments, the method includes introducing into the cell an agent that conditionally increases transcription of the coding sequence. In some embodiments, the agent activates a conditional promoter system operably linked to the coding sequence. In other embodiments, the method includes introducing into the cell a nucleic acid vector including an expression system having a conditional promoter system operably linked to the coding sequence. In further embodiments, the conditional promoter system includes a tetracycline inducible promoter.

Another feature of the present invention provides a method for activating a cell in a host by administering to the host an effective amount of an agent that conditionally increases transcription of a coding sequence of either TERT or TERC to activate the cell. In some embodiments, the subject method conditionally increases transcription of a TERT coding sequence. In other embodiments, the subject method conditionally increases transcription of a TERC coding sequence. Such a cell includes a hair follicle cell; a pancreatic islet cell; a neuronal cell; a bone marrow cell; and the like. The methods may be performed in vitro or in vivo. In some embodiments, the cell is present in a mammal, such as a human.

In some embodiments, the method includes introducing into the cell a nucleic acid vector including an expression system having a conditional promoter system operably linked to the coding sequence. In further embodiments, the conditional promoter system includes a tetracycline inducible promoter.

Yet another feature of the invention provides a method for activating a hair follicle cell in a host in vivo by administering to the host an effective amount of an agent that conditionally increases transcription of a coding sequence of either TERT or TERC to activate the hair follicle cell. In some embodiments, the activation of the hair follicle cells results in hair growth.

In some embodiments, the subject method conditionally increases transcription of a TERT coding sequence. In other embodiments, the subject method conditionally increases transcription of a TERC coding sequence. The methods may be performed in vitro or in vivo. In some embodiments, the cell is present in a mammal, such as a human. In some embodiments, the method includes introducing into the cell a nucleic acid vector including an expression system having a conditional promoter system operably linked to the coding sequence. In further embodiments, the conditional promoter system includes a tetracycline inducible promoter.

Yet another feature of the invention provides a transgenic animal, wherein the transgenic animal conditionally transcribes either TERT or TERC. In some embodiments, the transgenic animal includes a TERT transgene. In other embodiments, the transgenic animal includes a TERC transgene. In such embodiments, the transgenic animal is a mammal, such as a rodent.

In some embodiments, the conditional transcription is provided by a conditional promoter system operably linked to the TERT transgene or TERC transgene. In further embodiments, the conditional promoter system is a tetracycline inducible promoter system.

Yet another feature of the invention provides a method for identifying a compound that is capable of modulating the activity of one of TERT or TERC, by activating a cell by conditionally increasing transcription of a coding sequence of either TERT or TERC; administering a compound to the cell; and observing the effect of the compound on the cell. In some embodiments, the activating includes conditionally increasing transcription of a TERT coding sequence. In other embodiments, the activating includes conditionally increasing transcription of a TERC coding sequence. In such methods, the cell may be in a mammal, such as rodent, such as a mouse. In such methods, the compound may be a polypeptide, a nucleic acid, or a small molecule. In such methods, the modulating may be enhancing activity or repressing activity. In such embodiments, such activity may include active extension of telomeric repeat sequences at the ends of chromosomes, or may not include active extension of telomeric repeat sequences at the ends of chromosomes.

In some embodiments, the activating includes administering to the cell an agent that conditionally increases transcription of the coding sequence. In further embodiments, the activating includes administering an agent that activates a conditional promoter system operably linked to the coding sequence. In other embodiments, method further includes introducing into the cell a nucleic acid vector including an expression system having a conditional promoter system operably linked to the coding sequence. In further embodiments, the conditional promoter system includes a tetracycline inducible promoter.

Yet another feature of the invention provides a system for use in identifying a compound that is capable of modulating the activation of either TERT or TERC, including transgenic animal conditionally transcribing either TERT or TERC, and an agent that activates conditional transcription of the transgene. In some embodiments, the conditional transcription is provided by a conditional promoter system operably linked to the TERT transgene or TERC transgene. In further embodiments, conditional promoter system is the tetracycline inducible promoter system. In such systems, the animal may be a mammal, such as rodent, such as mouse. In addition, in such systems, the agent may be doxycycline or an analog thereof.

Yet another feature of the invention provides a conditional expression vector including a conditional promoter system operably linked to the coding sequence of either TERT OR TERC. In some embodiments, the conditional promoter system is a tetracycline inducible promoter system.

Yet another feature of the invention provides a system for use in producing a conditional expression animal model including a conditional expression vector that includes a conditional promoter system operably linked to the coding sequence of either TERT or TERC, and an animal. In some embodiments, the conditional promoter system is a tetracycline inducible promoter system. In further embodiments, the animal is a mammal, such as rodent.

### BRIEF DESCRIPTION OF THE DRAWINGS

The patent or application file contains at least one drawing executed in color. Copies of this patent or application publication with color drawing(s) will be provided by the U.S. Patent and Trademark Office upon request and payment of necessary fee.

The invention is best understood from the following detailed description when read in conjunction with the accompanying drawings. It is emphasized that, according to common practice, the various features of the drawings may not be to scale. On the contrary, the dimensions of the various features may be arbitrarily expanded or reduced for clarity. Included in the drawings are the following figures:

FIG. 1A is a schematic depiction of actin-rtTA and tetop-TERT transgene constructs.

FIG. 1B is an image of a Northern blot showing expression of TERT mRNA in the skin of i-TERT Tg treated with doxycycline (dox) mice, but not in i-TERT Tg (−dox) mice or non-transgenic littermates (WT) at day 50.

FIG. 1C is an image showing the induction of telomerase activity in the skin of i-TERT Tg (+dox) mice as compared with i-TERT Tg mice (−dox) or WT mice at day 50.

FIG. 1D is a diagram of anagen and telogen hair follicle cycle.

FIG. 1E is an image showing that telomerase activity is high during the anagen phase of the hair follicle and silenced during catagen and telogen phases in hair follicle cycling. Extracts are taken from skin at postnatal days 4 and 10 (anagen), 16 (catagen), 19 and 21 (telogen), 28 (anagen), 34 (catagen), and 52 (telogen).

FIG. 1F is a photograph of i-TERT Tg mouse (+dox) (background) and i-TERT Tg (−dox) (foreground) at day 50, showing the disorganized fur and droopy whiskers of the +dox mouse.

FIG. 1G is a histological analysis showing that TERT activation, beginning at day 21, promotes changes in the state of the hair follicle from telogen to anagen at day 50. Follicles were appropriately in anagen at day 28 in both groups. i-TERT Tg (−dox) mice were indistinguishable from non-transgenic mice.

FIG. 1H shows immunofluorescence sections of hair follicle epithelium skin of i-TERT Tg mice from day 50 following induction of TERT mRNA by doxycycline treatment. Merging of the immunofluorescence images shows an overlap in distribution pattern of TERT with keratin-14 protein.

FIGS. 2A-2H shows intact differentiation and development in TERT induced hair follicles. In each panel, TERT-induced anagen (day 50), denoted Tg(+dox), is compared to non-transgenic anagen (day 28) and age-matched non-transgenic mice in telogen (day 50). Immunofluorescence showed normal patterns of: outer root sheath differentiation by keratin-14 staining (FIG. 2A); inner layer of outer root sheath differentiation marked by keratin-6 (FIG. 2B); hair differentiation by AE13 staining (FIG. 2C); Normal inner root sheath differentiation marked by AE15 (FIG. 2D); proliferation in the matrix cells by Ki-67 staining (FIG. 2E). In situ hybridization analysis showed: normal, asymmetic pattern of Shh expression in the invaginating anagen hair follicle in both WT (day 28) and i-TERT Tg (day 50) (FIG. 2F); Lef1 is expressed in the matrix cells in both the WT and i-TERT Tg induced anagen hair follicle, but is absent from the telogen hair follicle (FIG. 2G); and Shh is absent from normal telogen (WT day 50) (FIG. 2H).

FIGS. 3A-3C shows that TERT triggers a rapid transition from telogen to anagen. i-TERT Tg mice and non-transgenic littermates (WT) were treated with doxycycline beginning at day 40, monitored through serial biopsies 0, 3, 9 and 12 days subsequently (day 0, 3, 9, 12). FIG. 3A shows that TERT mRNA expression was first detected at day 3, but increased substantially by day 9 via Northern blot (left). GAPDH was used as a loading control. Telomerase activity increased with similar kinetics seen by TRAP assay (right). FIG. 3B is histological data from the WT and iTERT TG groups showing that both groups were in telogen phase at the initiation of the experiment, age 40 days (day 0). After 9 days on doxycycline, follicles in i-TERT Tg mice entered early anagen (arrow), whereas controls remained in telogen (asterisk). Full anagen occured by 12 days on doxycycline in i-TERT mice. H&E, 20×. FIG. 3C is a photograph of mice that were administered doxycycline in telogen at age 45 days, shaved at age 55 days, and monitored for 14 days. Shaved hair briskly grew only in i-TERT Tg mice (+dox) (right), but not in i-TERT Tg mice (−dox) (middle) or non-transgenic littermates (left).

FIGS. 4A-4B shows that TERT activates hair follicle stem cells independent of its function in telomere synthesis. TERC+/− mice were backcrossed to the FVB/N strain, then intercrossed with i-TERT Tg mice to generate cohorts of i-TERT Tg mice on TERC+/+, TERC+/− or TERC−/− backgrounds. Mice in each group were treated with doxycycline beginning at day 21 and analyzed at day 50. FIG. 4A is histological analysis showing that induction of TERT facilitated transition from telogen to anagen in all TERC backgrounds, including TERC+/+, TERC+/−, and TERC−/−. Negative controls remained in telogen including, i-TERT (−dox), single transgenic mice, and non transgenic mice in TERC+/+, TERC+/−, and TERC−/− backgrounds. FIG. 4B shows that skin samples from i-TERT Tg and TERC−/− mice lacked telomerase activity by TRAP and TERC expression by RT PCR. The TERT transgene was induced similarly in i-TERT Tg mice, irrespective of TERC genotype.

FIGS. 5A-5C shows that telomeres remain stable and capped in i-TERT Tg mice. FIG. 5A is a northern analysis showing induction of Tert in i-Tert Tg MEFS treated with doxycycline for 72 hours (left) or splenocytes treated with doxycycline for 48 hours (right) as compared with controls. FIG. 5B shows images of metaphase preparations from MEFs (left) and splenocytes (right), which showed no increase in chromosomal end-to-end fusions with TERT induction. FIG. 5C is a table depicting the average number of chromosomes, and number of fusions per metaphase found in WT, i-TERT Tg(−dox), and i-Tert Tg(+dox) samples. No fusions were found in any metaphases.

FIGS. 6A-6D shows that induction of TERT does not lead to increased apoptosis or anaphase bridge formation. FIG. 6A shows the results of a TUNEL assay that was performed on skin sections from i-Tert Tg(+dox) mice at day 50 as well as WT at day 50, WT at day 28, and late generation Tert−/− at day 28 as controls. Increased number of TUNEL+ cells were only detected in the late generation Tert−/− sections. Anaphase bridges were detected in late generation Tert−/− skin sections but not in the i-Tert Tg(+dox) skin sections or WT controls. FIG. 6B is a bar graph depicting the average number of TUNEL positive cells per hair follicle. FIG. 6C is a bar graph depicting the number of anaphase bridges per total number of anaphases surveyed. FIG. 6D is a table indicating the number of anaphases surveyed and the fraction that were bridges in each genotype. Anaphase bridges were only found in the late generation Tert−/− skin sections.

FIGS. 7A-7B shows the conditional activation of TERC and the analysis f the hair follicle. FIG. 7A is a schematic depiction of actin-rtTA and tetop-TERC transgene constructs. FIG. 7B shows the results of a histological analysis from 50 day old mice showing that TERC activation promotes changes in the state of the hair follicle from telogen to anagen in the TERC Tg mice (+dox) (bottom) as compared to the TERC Tg (−dox) (middle) and non-trangenic littermates (top).

FIG. 8 is a photograph of mice that were administered doxycycline in telogen at age 45 days, shaved at age 55 days, and monitored for 14 days. Shaved hair briskly grew in iTERT Tg mice (+dox) (right) and iTERC Tg mice (+dox) (middle), but not in the wild type (non transgenic) littermates (left).

FIG. 9 shows tissue sections from i-TERC mice on doxycycline (right panel) and wild type controls (left panel) were hybridized with an anti-sense TERC probe. As shown in FIG. 9, transgenic TERC (red) was detected in the skin epithelium, in a pattern that overlaps with keratin-14 (green), a marker of the basal layer of the epidermis and the outer root sheath of the hair follicle.

FIGS. 10A-10F shows that TERT activates stem cells, depleting BrdU label from LRCs. FIG. 10A shows the maintenance of immunofluorescence for BrdU (red) and CD34 (green) of LRCs in Non-Tg group, but dramatic loss of BrdU label in i-TERT mice after doxy treatment (pre-doxy=day 55, post-doxy=day 90). FIG. 10B is a graph showing the quantification of LRC data from FIG. 10A. The graph shows that the number of BrdU+ cells/CD34+ cells. i-TERT (black bars, n=4 mice), Non-Tg (open bars, n=3 mice), (−) indicates pre-doxy, (+) indicates post-doxy. FIG. 10C is an LRC analysis from whole mounts of epidermis from tail of mice labeled with BrdU at day 10, switched to doxy at day 40 and analyzed at day 65. (BrdU=red, K14=green). FIG. 10D shows immunofluorescence using Ki-67 (red) to mark proliferating cells and K14 (green) to identify basal layer of skin. FIG. 10E is a graph showing the quantitation of proliferation index in FIG. 10D as Ki-67+ cells/100 μm length of basal layer (n=2 mice for each comparison). FIG. 10F shows a GFP epifluorescence costained with CD34 (inset, confocal microscopy) in skin section from an actin-GFP mouse. FIG. 10G shows RNA in situ analysis for TERT mRNA in i-TERT(+doxy) mouse skin. The inset shows TERT mRNA expression (cytoplasmic) overlaps in bulge with LRCs, marked by BrdU (nuclear). FIG. 10H shows H&E sections from K5tTA+; tetop-TERT+ (−doxy) (bottom) and Non-Tg (top) mice, 20×. Error bars indicate standard deviation. p values based on t-test. *=autofluorescence of hair.

### DETAILED DESCRIPTION OF THE INVENTION

Methods and compositions for cell activation are provided. In practicing the subject methods, transcription of a coding sequence for either (i.e., one of) a telomerase reverse transcriptase (TERT) or a telomerase RNA component (TERC) is conditionally increased. Also provided are transgenic animals and systems for practicing the subject methods.

Before the present invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a cell” includes a plurality of such cells and reference to “the agent” includes reference to one or more agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Furthermore, the dates of publication provided may be different from the actual publication dates which may need to be independently confirmed.

The practice of the present invention will employ, unless otherwise indicated, conventional methods of chemistry, biochemistry, recombinant DNA techniques and virology, within the skill of the art. Such techniques are explained fully in the literature. See, e.g., Fundamental Virology, 2nd Edition, vol. I & II (B. N. Fields and D. M. Knipe, eds.); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.); Oligonucleotide Synthesis (N. Gait, ed., 1984); A Practical Guide to Molecular Cloning (1984).

Methods

As summarized above, the subject invention provides a method for activating a cell. By “activating” is meant that the cell state of the cell is progressed or transitioned from a first, quiescent state to a second non-quiescent state. As used herein a “quiescent state” means a non-proliferating and non-transcriptionally active state, i.e., a state in which the cellular number of one or more cells is not increasing by cellular division, or increasing at a level below that of an actively proliferating state. As used herein a “non-quiescent state” means either a proliferating state, i.e., a state in which the cellular number of one or more cells is increasing by cellular division, or a non-proliferating and transcriptionally active state, i.e., a state in which the transcription rate of nucleic acid coding sequences within the cell is increased, e.g., by at least about 2-fold, as compared to the first non-transcriptionally active state, and where the cellular number of one or more cells is not increasing by cellular division, or increasing at a level below that of an actively proliferating state. The “non-quiescent state” may include active extension of telomeric repeat sequences at the ends of chromosomes, or may not include active extension telomeric repeat sequences at the ends of chromosomes. In other words, “activating” a cell by the subject method to a second “non-quiescent state” does not require that active extension of telomeric repeat sequences at the ends of chromosomes occur during the second “non-quiescent state”.

In some embodiments, the subject method provides for activating a cell by progressing or transitioning a cell from a first state of non-proliferation to a second state of proliferation, wherein by a second state of proliferation is meant that the cellular number is increasing by cellular division as compared to the first state of non-proliferation. In further embodiments, the second state of proliferation also includes active extension of telomeric repeat sequences at the ends of chromosomes. In other embodiments, the second state of proliferation does not include active extension of telomeric repeat sequences at the ends of chromosomes.

In addition, with respect to undedicated progenitor cells (i.e., undifferentiated stem cells), by activating is meant that the progenitor cell is moved from a first quiescent state to second non-quiescent state, where the first quiescent state is characterized by a state in which the cellular number is not increasing by cellular division, or increasing at a level below that of an actively proliferating state, and the second non-quiescent state is characterized by a state in which the cellular number is increasing by cellular division as compared to the first quiescent state, and the cellular progeny resulting from the cellular division develop into cells that further differentiate into specific cell types with distinctive characteristics as compared to the undedicated progenitor cells. As used herein “proliferating” refers to the ability of a target cell to undergo cellular division where the daughter cells of such divisions are not transformed, i.e., they maintain normal response to growth and cell cycle regulation. In such embodiments, the second non-quiescent state may also include active extension of telomeric repeat sequences at the ends of chromosomes, or may not include active extension of telomeric repeat sequences at the ends of chromosomes.

In further embodiments, with respect to undedicated progenitor cells (i.e., undifferentiated stem cells), by activating is meant that the progenitor cell is moved from a first quiescent state to second non-quiescent state, where the first quiescent state is characterized by a state in which the cellular number is not increasing by cellular division, or increasing at a level below that of an actively proliferating state, and the second non-quiescent state is characterized by a state of self-renewal. By “self-renewal” is meant that the cellular number of the progenitor cell is increasing by cellular division as compared to the first quiescent state, and the cellular progeny resulting from the cellular division are not more developed, i.e., further differentiated into specific cell types with distinctive characteristics, as compared to the parent undedicated progenitor cells. In such embodiments, the second non-quiescent state may also include active extension of telomeric repeat sequences at the ends of chromosomes, or may not include active extension of telomeric repeat sequences at the ends of chromosomes.

In other embodiments, the subject method provides for activating a cell by progressing or transitioning a cell from a first non-transcriptionally active state to a second transcriptionally active state, wherein by a second transcriptionally active state is meant that the transcription rate of nucleic acid coding sequences within the cell is increased as compared to the first non-transcriptionally active state, and where the cellular number of one or more cells is not increasing by cellular division, or increasing at a level below that of an actively proliferating state. In such embodiments, the second transcriptionally active state may also include active extension of telomeric repeat sequences at the ends of chromosomes, or may not include active extension of telomeric repeat sequences at the ends of chromosomes.

In certain embodiments in which the subject method provides for activating a cell by progressing or transitioning a cell from a first non-proliferating state to a second proliferating state, activation of a target cell can be determined by detecting an increase in the proliferative capacity of the target cell. The term “proliferative capacity” as used herein refers to the number of cellular divisions that a cell can undergo in response to a stimulus. In such embodiments an increase in the proliferative capacity of a target cell means an increase of at least about 1.2 to about 2 fold, usually at least about 5 fold and often at least about 10, 20, 50 fold or even higher, compared to a control. A suitable control for use in such methods is an untreated or mock-treated target cell, where the mock-treated cell is exposed to the same conditions as the treated target cell. Methods for measuring cellular proliferation are well known in the art and can be used in with the subject methods to assess activation of target cell in response to the subject methods.

Methods for measuring cell activation may be direct, such that the increase in actual daughter cells of the target cells are detected in the treated target cells as compared to control cells. In addition, methods for measuring cell activation may be indirect, e.g., such that an increase in cellular division mediating proteins are detected, or a decrease in cell cycle inhibitor proteins is detected in the treated target cells as compared to control cells.

In some embodiments, an increase in the proliferative capacity of a target cell may be determined by measuring the incorporation of a labeled nucleotide into the newly synthesized DNA of daughter cells during cellular division. Cells incorporate the labeled DNA precursors into newly synthesized DNA, such that the amount of incorporation in the treated target cell as compared to control cells is a relative measure of cellular proliferation. A labeled nucleotide suitable for use with such assays includes, but is not limited to, a radio-labeled nucleotide, such as [³H]-thymidine or [¹⁴]-thymidine, where the incorporation of the radio-labeled nucleotide may be measured by liquid scintillation counting.

In other embodiments, an increase in the proliferative capacity of a target cell may be determined by measuring the incorporation of a fluorescent dye into the membranes of daughter cells of treated target cells. For example, an aliphatic reporter molecule that acts as a plasma membrane dye and is incorporated into the plasma membranes of the daughters of replicating cells can be used to measure the relative number of daughter cells of treated target cells as compared to control cells. An example of such a cellular proliferation assay is the Cell Census Plus™ System (Sigma-Aldrich, St. Louis, Mo.) as described in U.S. Pat. Nos. 4,783,401; 4,762,701; 4,859,584, incorporated here by reference.

In yet other embodiments, an increase in the proliferative capacity of a target cell may be determined by measuring an increase in the activity or the expression of cellular division mediating proteins, or a decrease in the activity or expression of cell cycle regulator proteins, such as cyclin-dependent kinase (CDK), in the treated target cells as compared to control cells. For example, a cyclin-dependant kinase assay may be used to measure the change in activity of treated target cells as compared to control cells. In addition, methods such as Western blot, ELISA, or immunocytochemistry can be used to quantify expression levels of such proteins in order to determine the proliferative capacity of a target cell.

In certain embodiments in which the subject method provides for activating a cell by progressing or transitioning a cell from a first non-transcriptionally active state to a second transcriptionally active state, cell activation may be determined by for example; and not limited to, measuring an increase or in the activity of transcription factors, an increase in the transcription of target nucleic acids, or a decrease in the activity of transcription repressors in the treated target cells as compared to control cells.

In some embodiments, an increase in the transcriptional activity of a target cell may be detecting an increase in the transcription of target nucleic acids in the treated target cells. For example, the coding sequence for a detectable protein, such as green-fluorescent protein or luciferase, may be used to detect activation of a treated target cell as compared to a control cell. In such embodiments an increase in transcription means an increase of at least about 1.2 to about 2 fold, usually at least about 5 fold and often at least about 10, 20, 50 fold or even higher, compared to a control. A suitable control for use in such methods is an untreated or mock-treated target cell, where the mock-treated cell is exposed to the same conditions as the treated target cell.

In some embodiments, an increase in the transcriptional activity of a target cell may be detecting the level of translocation of transcription factors in the treated target cells. For example, the level of translocation of a transcription factors, such as NF-κB, from the cytoplasm to the nucleus can be used to detect cell activation of a target treated cell as compared to a control cell. In such embodiments an increase in the level of translocation of a transcription factors from the cytoplasm to the nucleus means an increase of at least about 1.2 to about 2 fold, usually at least about 5 fold and often at least about 10, 20, 50 fold or even higher, compared to a control. A suitable control for use in such methods is an untreated or mock-treated target cell, where the mock-treated cell is exposed to the same conditions as the treated target cell.

As such, in certain embodiments, the subject methods provide for activation of a specific dedicated cell (i.e., non-progenitor cell), from a first quiescent, non-proliferating state, to a second non-quiescent, proliferating state, wherein the second non-quiescent, proliferating state is characterized by an increase in cellular number resulting from cellular division, as compared to the first quiescent, non-proliferating state.

In other embodiments, the subject methods provide for activation of a progenitor cell (i.e., non-dedicated cell), from a first quiescent, non-proliferating state, to a second non-quiescent, proliferating state, where the second non-quiescent, proliferating state is characterized by an increase in cellular number resulting from cellular division, as compared to the first quiescent state, and the cellular progeny resulting from the cellular division develop into cells that further differentiate into specific cell types with distinctive characteristics as compared to the undedicated progenitor cells

In such methods, a cell is activated by conditionally increasing transcription (e.g., expression) of a coding sequence of either (e.g., only one of) a telomerase reverse transcriptase component (TERT) or a telomerase RNA component (TERC) in a manner sufficient to activate the cell. The subject methods of the present invention can be performed in vitro, where activation of the cells is achieved ex vivo in for example, tissue culture, or the methods can be performed in vivo, where activation of cells in achieved in an organism.

Thus, in one aspect the subject methods of the present invention provide for cell activation by conditionally increasing transcription (e.g., expression) of a TERT coding sequence. TERT is the catalytic protein component of telomerase. In some embodiments, a TERT coding sequence suitable for use in the subject methods is human TERT (hTERT). The coding sequence for hTERT is provided in Genbank Accession Nos. AF114847 and AF128893, and is further described in U.S. Pat. No. 6,166,178, incorporated herein by reference.

In another aspect the subject methods of the present invention provide for cell activation by conditionally increasing transcription (e.g., expression) of a TERC coding sequence. TERC acts as a template for the addition of telomeric repeat sequences at the ends of chromosomes by telomerase. In some embodiments, a TERC coding sequence suitable for use in the subject methods is human TERC (hTERC). The coding sequence for hTERC is provided in Genbahk Accession No. AF7544491, and is further described in Feng et al., 1995, Science 269:1236-1241.

The subject methods of activating a cell can be performed by introducing into a cell an agent that conditionally increases transcription of a coding sequence of either TERT or TERC. As such, in some embodiments, the subject method is achieved by contacting a cell (e.g., through administration to a host or subject that includes the cell) with an effective amount of an agent that conditionally increases transcription of an endogenous coding sequence for either TERT, or a fragment thereof, or TERC, or a fragment thereof, present in the genome of the subject cell. In such embodiments, the conditionally expressed TERT or TERC may be capable of extension of telomere ends, or may not be capable of extension of telomere ends.

In other embodiments, the subject method is achieved by introducing into a cell (e.g., through administration to a host or subject that includes the cell) a nucleic acid composition that encodes the coding sequence of either TERT, or a fragment thereof, or TERC, or a fragment thereof operably linked to a conditional promoter system. In such embodiments, the conditionally expressed TERT or TERC may be capable of extension of telomere ends, or may not be capable of extension of telomere ends.

By “conditional” is meant that the level of transcription of a coding sequence is modulated by the presence of an active regulatory agent, wherein the presence of the active regulatory agent either increases or decreases the level of transcription of the coding sequence, as compared to the level of transcription of the coding sequence in the absence of the active regulatory agent. In other words, the transcription of a coding sequence is conditional on the presence of an active regulatory agent, wherein the agent itself either directly increases transcription or indirectly increases transcription, e.g., by interacting and muting a repressive agent that acts by decreasing or repressing transcription of the coding sequence. As such, conditional is the opposite of “constitutive” as that term is used in the art, i.e., to refer to a gene which is continuously expressed without any regulation (transcription can be neither suppressed nor encouraged).

By “increasing the transcription of a coding sequence” is meant that the level of transcription of the coding sequence is increased by at least about 2 fold, usually by at least about 5 fold and sometimes by at least 25, 50, 100, 150, 200 fold and in particular about 300 fold higher, as compared to a control, i.e., transcription from an expression system that is not subjected to the methods of the present invention, or as compared to transcription level of the coding sequence in the absence of the active regulatory agent. Alternatively, in cases where transcription of the coding sequence in the absence of the active regulatory agent is so low that it is undetectable transcription of the coding sequence is considered to be increased in the presence of the active regulatory agent if transcription is increased to a level that is easily detected.

As mentioned above, the subject methods can be achieved by introducing into the target cell an agent that conditionally increases transcription of an endogenous coding sequence for one of TERT or TERC. By endogenous is meant the naturally existing coding sequence present in the genomic DNA of the target cell. As such, in some embodiments the agent acts by inhibiting the repression of transcription from the coding sequence of one of TERT or TERC. By inhibition of repression is meant that the repressive activity of a TERT or TERC coding sequence repressor binding site or repressor protein interaction with respect to TERT or TERC transcription is decreased by a factor sufficient to at least provide for the desired enhanced level of TERT or TERC transcription, as described above. Inhibition of transcription repression may be accomplished in a number of ways, where representative protocols for inhibiting TERT or TERC transcription repression are provided below.

One representative method of inhibiting repression of transcription is to employ double-stranded, i.e., duplex, oligonucleotide decoys for the repressor protein, which decoys bind to the repressor protein and thereby prevent the repressor protein binding to its target site in the TERT or TERC promoter. Such duplex oligonucleotide decoys have at least a portion of the sequence of a repressor site required to bind to the repressor protein and thereby prevent binding of the repressor protein to the repressor site. In many embodiments, the length of such duplex oligonucleotide decoys ranges from about 5 to about 5000, usually from about 5 to about 500 and more usually from about 10 to about 50 bases. In using such oligonucleotide decoys, the decoys are placed into the environment of the repressor site and its repressor protein, resulting in de-repression of the transcription of the TERT or TERC coding sequence. Oligonucleotide decoys and methods for their use and administration are further described in general terms in Morishita et al., Circ Res (1998) 82 (10):1023-8.

Instead of the above-described decoys, other agents that disrupt binding of a repressor protein to the target repressor binding site and thereby inhibit its transcription repression activity may be employed. Other agents of interest include, among other types of agents, small molecules that bind to the repressor protein and inhibit its binding to the repressor region. Alternatively, agents that bind to the repressor sequence and inhibit its binding to the repressor protein are of interest. Alternatively, agents that disrupt repressor protein-protein interactions with cofactors, e.g., cofactor binding, and thereby inhibiting repression are of interest.

Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below. Small molecule agents of particular interest include pyrrole-imidazole polyamides, analogous to those described in Dickinson et al., Biochemistry 1999 Aug. 17; 38(33):10801-7. Other agents include “designer” DNA binding proteins that bind the repressor site (without causing repression) and prevent the repressor proteins from binding.

In yet other embodiments, expression of the repressor protein is inhibited. Inhibition of repressor protein expression may be accomplished using any convenient means, including administration of an agent that inhibits repressor protein expression (e.g., antisense agents), inactivation of the repressor protein gene, e.g., through recombinant techniques, etc.

The anti-sense reagent may be antisense oligodeoxynucleotides (ODN), particularly synthetic ODN having chemical modifications from native nucleic acids, or nucleic acid constructs that express such anti-sense molecules as RNA. The antisense sequence is complementary to the mRNA of the targeted repressor protein, and inhibits expression of the targeted repressor protein. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

As also noted above, the subject methods can be achieved by introducing into the target cell a nucleic acid composition, e.g., a nucleic acid vector including an expression system, where the nucleic acid composition includes a coding sequence for one of TERT or TERC. Conditional regulation of a coding sequence may be achieved by placing the coding sequence under conditional regulation of a conditional promoter system, such that there is no, or an undetectable level, of transcription of the coding sequence in the absence of an active regulatory agent (e.g., a molecule) that regulates transcription of the coding sequence through the conditional promoter system. As such, the active regulatory agent regulates transcription of the coding sequence through the conditional promoter system.

A suitable conditional promoter system for use with the subject methods of the invention is any sequence that may be regulated to alter transcription of an associated coding sequence. A conditional promoter system may be capable of regulating gene transcription at any step, including, for example, transcription initiation, transcription elongation, transcription termination, mRNA stability, RNA splicing, and translation.

Regulatory agents and molecules that control gene transcription are well known in the art. Regulatable gene transcription inhibitor elements are generally targets for regulation by a corresponding regulatory agent or compound. For example, regulatable gene transcription inhibitor elements include transcription termination sequences, transcription factor binding sites, ribozyme target sites, splice acceptor sites, dsRNAi target sequences, short interfering RNA (siRNA) target sequences, short hairpin RNA (shRNA) target sequences, and antisense. RNA targets. Regulatable gene transcription inhibitor elements of the invention may mediate a reduction in transcription of an associated coding sequence in the presence of a corresponding regulatory molecule or compound. Alternatively, gene transcription inhibitor elements of the invention mediate a reduction in expression of an associated gene-upon removal of a regulatory compound.

Regulatory agents and compounds include any molecule or compound capable of regulating gene expression via the regulatable gene expression inhibitor element, either directly or indirectly. In certain embodiments, the active regulatory agent conditionally increases transcription of the coding sequence by directly interacting with the conditional promoter system, thereby increasing transcription. In other embodiments, the active regulatory agent conditionally increases transcription of the coding sequence by indirectly interacting with the conditional promoter system, wherein the indirect interaction with the conditional promoter system is by directly interacting with an agent that is repressing (e.g., inhibiting) transcription from the conditional promoter system. In such embodiments, the active regulatory agent increases transcription of the coding sequence by interacting with the repressive agent, thereby dissociating the repressive agent form the conditional promoter system and allowing transcription of the coding sequence. For example, a regulatory agent may be a binding partner for a molecule that interacts with the regulatable gene expression inhibitor element, or a regulatory agent may promote the release of an inhibitory molecule from a molecule that binds a regulatable gene expression inhibitor element. A regulatory agent may also, e.g., act by activating a second molecule that acts on the regulatable gene expression inhibitor element, or by altering subcellular localization of a molecule that acts directly on the regulatable gene expression inhibitor element.

In certain embodiments, the conditional promoter system suitable for use with the subject methods of the invention is the Ecdysone-Inducible Expression System (Invitrogen). The Ecdysone-Inducible expression system uses the steroid hormone ecdysone analog, muristerone A, to activate expression of a operably linked coding sequence via a heterodimeric nuclear receptor (No et al., 1996, PNAS, 93:3346). In such embodiments, a coding sequence for one of TERT or TERC polypeptide is cloned into an expression vector, which the expression vector contains five modified ecdysone response elements (E/GREs) upstream of a minimal heat shock promoter and the multiple cloning site. Conditional transcription from the expression vector is then induced with the administration of an activating agent to the target cells. In such embodiments the activating agent suitable fir use with the ecodysone-inducible expression system is muristerone A, wherein administration of muristerone A results in a conditional increase in transcription of the coding sequence.

In other embodiments, the conditional promoter system suitable for use with the subject methods is a tetracycline inducible promoter system, such as the Tet-On and Tet-off tetracycline regulated systems from Clontech. In such embodiment of the invention, a coding sequence for one of TERT or TERC polypeptide is conditionally transcribed using a tetracycline inducible promoter system, such as the Tet-on and Tet-off expression systems (Clontech) to provide regulated, high-level gene expression (Gossen et al., 1992, Proc. Natl. Acad. Sci. USA 89:5547; Gossen et al., 1995, Science 268:1766). In further embodiments, where the conditional promoter system is the a tetracycline inducible promoter system, such as the Tet-On and Tet-off tetracycline regulated systems, the active regulatory agent is tetracycline, doxicycline, or an analog thereof. The Tet-on and Tet-off expression system are further described in, for example, U.S. Pat. Nos. 5,464,758, 5,650,298, and 6,133,027, the disclosures of which herein incorporated by reference.

In yet other embodiments, the subject method is achieved by introducing into a cell (e.g., through administration to a host or subject that includes the cell) TERC ribonucleic acid, or a fragment, or mimetic thereof. In such embodiments, the introduction of the TERC ribonucleic acid, or a fragment, or mimetic thereof, may also be accompanied by the conditional expression of endogenous coding sequence for TERC, as further described above. In yet other embodiments, the subject method is achieved by introducing into a cell (e.g., through administration to a host or subject that includes the cell) polypeptides encoding TERT, or a fragment thereof.

The nucleic acids (e.g., expression vectors) for use in the subject methods of the invention may be introduced into a cell, tissue, organ, patient or animal by a variety of methods. The nucleic acid expression vectors (typically dsDNA) can be transferred into the chosen host cell by well-known methods such as calcium chloride transformation (for bacterial systems), electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223), agent-enhanced uptake of DNA, and ex vivo transduction. Useful liposome-mediated DNA transfer methods are described in U.S. Pat. Nos. 5,049,386, 4,946,787; and 4,897,355; PCT publications WO 91/17424, WO 91/16024; Wang and Huang, 1987, Biochem. Biophys. Res. Commun. 147: 980; Wang and Huang, 1989, Biochemistry 28:9508; Litzinger and Huang, 1992, Biochem. Biophys. Acta 1113:201; Gao and Huang, 1991, Biochem. Biophys. Res. Commun. 179:280. Immunoliposomes have been described as carriers of exogenous polynucleotides (Wang and Huang, 1987, Proc. Natl. Acad. Sci. U.S.A. 84:7851; Trubetskoy et al., 1992, Biochem. Biophys. Acta 1131:311) and may have improved cell type specificity as compared to liposomes by virtue of the inclusion of specific antibodies which presumably bind to surface antigens on specific cell types (Behr et al., 1989, Proc. Natl. Acad. Sci. U.S.A. 86:6982 report using lipopolyamine as a reagent to mediate transfection itself, without the necessity of any additional phospholipid to form liposomes.). Suitable delivery methods will be selected by practitioners in view of acceptable practices and regulatory requirements (e.g., for gene therapy or production of cell lines for expression of recombinant proteins). It will be appreciated that the delivery methods listed above may be used for transfer of nucleic acids into cells for purposes of gene therapy, transfer into tissue culture cells, and the like.

The subject nucleic acids may be produced using any convenient protocol, including synthetic protocols, e.g., those where the nucleic acid is synthesized by a sequential monomeric approach (e.g., via phosphoramidite chemistry); where subparts of the nucleic acid are so synthesized and then assembled or concatamerized into the final nucleic acid, and the like. Where the nucleic acid of interest has a sequence that occurs in nature, the nucleic acid may be retrieved, isolated, amplified etc., from a natural source using conventional molecular biology protocols.

Also provided are constructs comprising the subject nucleic acid compositions, e.g., those that include the coding sequence of one of TERT or TERC operably linked to a conditional promoter system, inserted into a vector, where such constructs may be used for a number of different applications, including cell activation as described herein. Constructs made up of viral and non-viral vector sequences may be prepared and used, including plasmids, as desired. The choice of vector will depend on the particular application in which the nucleic acid is to be employed. Certain vectors are useful for amplifying and making large amounts of the desired DNA sequence. Other vectors are suitable for expression in cells in culture, e.g., for use in screening assays. Still other vectors are suitable for transfer and expression in cells in a whole animal or person. The choice of appropriate vector is well within the skill of the art. Many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

In the subject methods, the active agent(s) may be introduced into to the targeted cells using any convenient means capable of resulting in the desired conditional enhancement of transcription of the coding sequence of one of TERT or TERC. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol; corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. oligonucleotide decoy, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

A variety of cells can be activated with the subject methods of the present invention, such as for example, but not limited to, hair follicle cells, pancreatic islet cells, neurons, and stem cells, such as for example, but not limited to, embryonic stem cells, embryonic germ cells, adult stem cells, fetal stem cells, bone marrow stem cells, and neuronal stem cells.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Practice of the subject methods, as described above, results in activation of a target cell or cells. The subject methods find use in a variety of different applications, representative applications of which are now reviewed in the following section of the application.

Utility

The subject methods of the present invention find use in a variety of applications in which the activation of a target cell is desired. As previously noted, activation of target cells according to the subject methods of the present invention find use in the treatment of disorders in which it is beneficial to progress a target cell from a first quiescent state to a second non-quiescent state. By treatment is meant at least an amelioration of the symptoms associated with the disease condition (or other target condition to be mediated) afflicting the host, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition. Disorders or conditions of interest include, but are not limited to, situations in which cells have become inactive (i.e., quiescent), as a result of a disease or premature cell cycle senescence, thereby resulting in an abnormal condition. Such conditions include, but are not limited to, hair loss as a result of hair follicle cell senescence, diabetic conditions as a result of decreased production of insulin by the pancreatic islet cells, neurodegenerative disorders, anemia, aplastic anemia, cancer, such as leukemia and myeloma, liver cirrhosis, degenerative joint disease, Alzheimer's disease, skin burns, wound healing, and the like.

In certain embodiments, the subject methods of the present invention find use in activation of hair follicle cells in order to progress the hair follicle cells from a first quiescent state to a second non-quiescent state, where the second non-quiescent state is characterized in an anagen growth phase, which anagen growth phase is results in hair growth. In such embodiments, activation of the hair follicle cells typically results in an increase in hair growth of at least 1.2 to about 2 fold, usually at least about 5 fold and often at least about 10, about 20, about 50 fold or even higher, compared to a control.

In other embodiments, the subject methods of the present invention find use in activation of pancreatic islet cells in order to progress the pancreatic islet cells from a first quiescent state to a second non-quiescent state, where the second non-quiescent state is characterized in an increase in cellular transcription activity of pancreatic polypeptides, such as insulin. In such embodiments, activation of the pancreatic islet cells typically results in an increase in hair growth of at least 1.2 to about 2 fold, usually at least about 0.5 fold and often at least about 10, about 20, about 50 fold or even higher, compared to a control, such as a target pancreatic islet cell that had not undergone activation according to the subject methods of the present invention.

In other embodiments, the subject methods of the present invention find use in activation of stem cells. In such embodiments, the subject methods find use in activation of stem cells in order to progress the stem cells from a first quiescent state to a second non-quiescent state, where the second non-quiescent state is characterized in an increase in cellular proliferative capacity. In some embodiments, activation resulting in cellular proliferative capacity refers to the ability of the stem cells to undergo cellular division where the daughter cells of such divisions develop into cells that further differentiate into specific cell types and where such daughter cells are not transformed, i.e., they maintain normal response to growth and cell cycle regulation. In other embodiments, activation resulting in cellular proliferative capacity refers to the ability of the stem cells to undergo self-renewal, wherein self-renewal is an increase in the cellular number of the cell by cellular division as compared to the first quiescent state, and the cellular progeny resulting from the cellular division are not more developed, i.e., further differentiated into specific cell types with distinctive characteristics, as compared to the parent undedicated progenitor cells.

In such embodiments, an increase in proliferative capacity results in an increase in cellular division of at least 1.2 to about 2 fold, usually at least about 5 fold and often at least about 10, about 20, about 50 fold or even higher, compared to a control, such as a target neuronal stem cell that had not undergone activation according to the subject methods of the present invention.

In other embodiments, the subject methods of the present invention find use in activation of neuronal stem cells. In such embodiments, the subject methods find use in activation of neuronal stem cells in order to progress the neurons from a first quiescent state to a second non-quiescent state, where the second non-quiescent state is characterized in an increase in cellular proliferative capacity. In some embodiments, activation resulting in, cellular proliferative capacity refers to the ability of the neuronal stem cells to undergo cellular division where the daughter cells of such divisions develop into cells that further differentiate into specific cell types and where such daughter cells are not transformed, i.e., they maintain normal response to growth and cell cycle regulation. In other embodiments, activation resulting in cellular proliferative capacity refers to the ability of the neuronal stem cells to undergo self-renewal, wherein self-renewal is an increase in the cellular number of the cell by cellular division as compared to the first quiescent state, and the cellular progeny resulting from the cellular division are not more developed, i.e., further differentiated into specific cell types with distinctive characteristics, as compared to the parent undedicated progenitor cells.

In such embodiments; an increase in proliferative capacity results in an increase in cellular division of at least 1.2 to about 2 fold, usually at least about 5 fold and often at least about 10, about 20, about 50 fold or even higher, compared to a control, such as a target neuronal stem cell that had not undergone activation according to the subject methods of the present invention.

In other embodiments, the subject methods of the present invention find use in activation of bone marrow stem cells. In such embodiments, the subject methods find use in activation of bone marrow stem cells in order to progress the bone marrow stem cells from a first quiescent state to a second non-quiescent state, where the second non-quiescent state is characterized in an increase in cellular proliferative capacity. In some embodiments, activation resulting in cellular proliferative capacity refers to the ability of the bone marrow stem cells to undergo cellular division where the daughter cells of such divisions develop into cells that further differentiate into specific cell types and where such daughter cells are not transformed, i.e., they maintain normal response to growth and cell cycle regulation. In other embodiments, activation resulting in cellular proliferative capacity refers to the ability of the bone marrow stem cells to undergo self-renewal, wherein self-renewal is an increase in the cellular number of the cell by cellular division as compared to the first quiescent state, and the cellular progeny resulting from the cellular division are not more developed, i.e., further differentiated into specific cell types with distinctive characteristics, as compared to the parent undedicated progenitor cells.

In such embodiments, an increase in proliferative capacity results in an increase in cellular division of at least about 1.2 to about 2 fold, usually at least about 5 fold and often at least about 10, about 20, about 50 fold or even higher, compared to a control, such as a target bone marrow stem cell that had not undergone activation according to the subject methods of the present invention.

As indicated above, instead of a multicellular animal, the target may be a cell or population of cells, which are treated according to the subject methods and then introduced into a multicellular organism for therapeutic effect. As a non-limiting example of a target cell, the subject methods may be employed in bone marrow stem cell transplants for the treatment of anemia and cancer, such as leukemia and myeloma. In these cases, cells are isolated from a human donor and then cultured for transplantation back into human recipients. During the cell culturing, the cells normally age and senesce, decreasing their useful lifespans. Bone marrow cells, for instance, lose approximately 40% of their replicative capacity during culturing. This problem is aggravated when the cells are first genetically engineered (Decary, Mouly et al. Hum. Gene Ther. 7(11): 1347-50, 1996). In such cases, the therapeutic cells must be expanded from a single engineered cell. By the time there are sufficient cells for transplantation, the cells have undergone the equivalent of 50 years of aging (Decary, Mouly et al. Hum Gene Ther 8(12): 1429-38, 1997). Use of the subject methods spares the replicative capacity of bone marrow cells during culturing and expansion and thus significantly improves the survival and effectiveness of bone marrow transplants. In such embodiments, activation of the bone marrow stem cells may also include extension of telomeres, or such activation will not include extension of telomeres. In embodiments, where activation of the bona marrow stem cells does not include extension of telomeres, such activation is characterized by self-renewal of the stem cells. Any transplantation technology requiring cell culturing can benefit from the subject methods, including ex vivo gene therapy applications in which cells are cultured outside of the animal and then administered to the animal, as described in U.S. Pat. Nos. 6,068,837; 6,027,488; 5,824,655; 5,821,235; 5,770,580; 5,756,283; 5,665,350; the disclosures of which are herein incorporated by reference.

Screening Assays

Also provided by the subject invention are screening methods and assays for identifying compounds that are capable of modulating the activity of one of TERT or TERC, e.g., enhancing or repressing the activity of one of TERT or TERC. The conditions may be set up in vitro, e.g., in a cell that conditionally expresses the coding sequence for one of TERT or TERC, or in vivo, in an animal model that conditionally expresses the coding sequence of one TERT or TERC, as further described below. The screening methods may be an in vitro or in vivo format, where both formats are readily developed by those of skill in the art.

Whether the format is in vivo or in vitro, the target cell is first activated by conditionally increasing transcription of a coding sequence for either TERT or TERC, then the candidate agent is administered to the target cell, and the effect of the candidate agent on the target cell is observed. In such embodiments, the cell is activated by introducing into the target cell an agent that conditionally modulates (i.e., increases or decreases) transcription of an endogenous coding sequence for either TERT or TERC by decreasing inhibition of transcription of the coding sequence, as described above.

In some embodiments, the cell is activated by introducing into the target cell a nucleic acid expression system, e.g., a plasmid, that includes a coding sequence for one of TERT or TERC operably linked to conditional promoter system, as described above. As summarized above, following introduction of the nucleic acid expression system, the transcription of the TERT or TERC is conditionally increased by administering to the target cell an active regulatory agent. Once TERT or TERC transcription is conditionally increased, a candidate agent is administered to the cell and the effect of the administration of the candidate agent is observed on the target cells, as compared to control cells that were not administered the candidate agent.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents identified in the above screening assays that enhance the activity of one of TERT or TERC, by inhibiting the repression of TERT or TERC transcription find use in the methods described above, e.g., in the enhancement of TERT or TERC transcription. Alternatively, agents identified in the above screening assays that enhance the activity of one of TERT or TERC find use in applications where an increase in transcription of TERT or TERC, and the activation of the target cell is desired, e.g., in the treatment of disease conditions characterized by the senescence of the target cells, as described above.

Animal Models

Also provided by the subject invention are animal models for use in the subject screening methods described above. Such animal models for use in the subject screening methods are capable of activation of target cells by the conditional transcription of a coding sequence for either TERT or TERC.

In some embodiments, the conditional transcription animal model is capable of conditional transcription of a transgene, which transgene includes the coding sequence of either TERT or TERC. In further embodiments the conditional animal models of the present invention include a nucleic acid expression system, e.g., a plasmid, providing for the conditional transcription of TERT or TERc, where the nucleic acid vector includes the coding sequence for either TERT or TERC operably linked to a conditional promoter system, as described above. An example of a conditional promoter system suitable for use with the subject conditional transcription animal models is the tetracycline inducible promoter system, such as the Tet-On and Tet-off tetracycline regulated systems, where the active regulatory agent is tetracycline, doxicycline, or an analog thereof.

In other embodiments, the conditional transcription animal model is capable of conditional transcription of an endogenous coding sequence for either TERT or TERC. As further described above, the subject conditional transcription animal model can be achieved by introducing into the target cell of a subject animal an agent that conditionally increases transcription of an endogenous coding sequence for one of TERT or TERC. As such, in some embodiments the agent acts by inhibiting the repression of transcription from the coding sequence of one of TERT or TERC. By inhibition of repression is meant that the repressive activity of a TERT or TERC coding sequence repressor binding site or repressor protein interaction with respect to TERT or TERC transcription is decreased by a factor sufficient to at least provide for the desired enhanced level of TERT or TERC transcription, as described above. Inhibition of transcription repression may be accomplished in a number of ways, where representative protocols for inhibiting TERT or TERC transcription repression are provided in the above methods.

Examples of animals suitable for use include nonhuman animals such as apes, monkeys, pigs and rodents, such a rats, mice, and guinea pigs.

Systems

Also provided by the subject invention are systems for use in the subject screening methods described above. Such systems include at least a conditional transcription animal model that is capable of activation of target cells by the conditional transcription of the coding sequence for either TERT or TERC, as described above, and an agent that activates the conditional transcription of the coding sequence. An example of an animal suitable for use with the subject systems is a non-human animal, such as a rat, mouse, guinea pig, and the like.

In some embodiments, the conditional transcription animal model is capable of conditional transcription of a transgene, which transgene includes the coding sequence of either TERT or TERC. An example of a conditional promoter system suitable for use with the subject conditional expression vector is the tetracycline inducible promoter system, such as the Tet-On and Tet-Off tetracycline regulated systems, where the active regulatory agent is tetracycline, doxicycline, or an analog thereof.

In other embodiments, the conditional transcription animal model is capable of conditional transcription of an endogenous coding sequence for either TERT or TERC. As further described above, the subject conditional transcription animal model can be achieved by introducing into the target cell of a subject animal an agent that conditionally increases transcription of an endogenous coding sequence for one of TERT or TERC: As such, in some embodiments the agent that activates the conditional transcription of the coding sequence acts by inhibiting the repression of transcription from the coding sequence of one of TERT or TERC. By inhibition of repression is meant that the repressive activity of a TERT or TERC coding sequence repressor binding site or repressor protein interaction with respect to TERT or TERC transcription is decreased by a factor sufficient to at least provide for the desired enhanced level of TERT or TERC transcription, as described above. Inhibition of transcription repression may be accomplished in a number of ways, where representative protocols for inhibiting TERT or TERC transcription repression are provided in the above methods.

Also provided are systems that find use in producing a conditional expression animal model as described above. The systems for practicing the subject methods at least include a conditional expression vector, e.g., a plasmid, which vector includes a coding sequence for either TERT or TERC operably lined to a conditional promoter system; various buffers for use in carrying out the subject method of producing a conditional expression animal model; an animal; and the like. An example of a conditional promoter system suitable for use with the subject conditional expression vector is the tetracycline inducible promoter system, such as the Tet-On and Tet-Off tetracycline regulated systems, where the active regulatory agent is tetracycline, doxicycline, or an analog thereof. An example of an animal suitable for use with the subject systems is a non-human animal, such as a rat, mouse, guinea pig, and the like.

Furthermore, additional items that are required or desired in the protocol to be practiced with the system components may be present, which additional items include, but are not limited to: means for delivering the expression vector to the animal, e.g. a syringe; one or more reagents necessary for preparation of the conditional expression animal model, such as reagents necessary for the induction of the expression vector into the animal, and the like; and instructions for carrying out the subject methods.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following materials and methods are used in the examples below.

Transgenic Mice

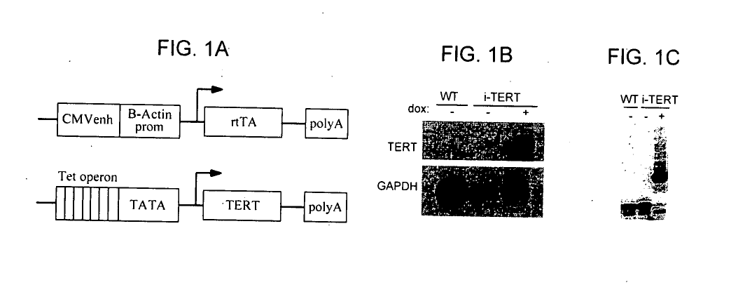

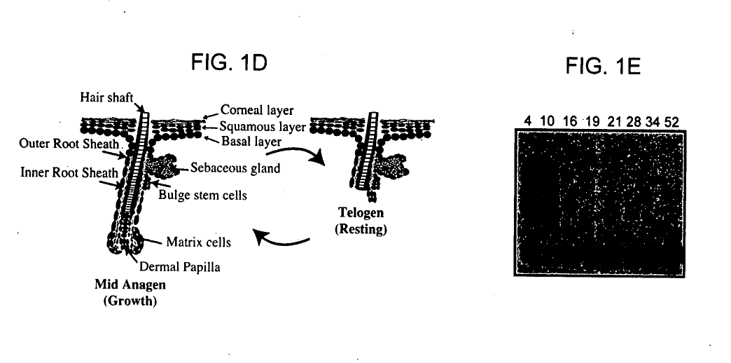

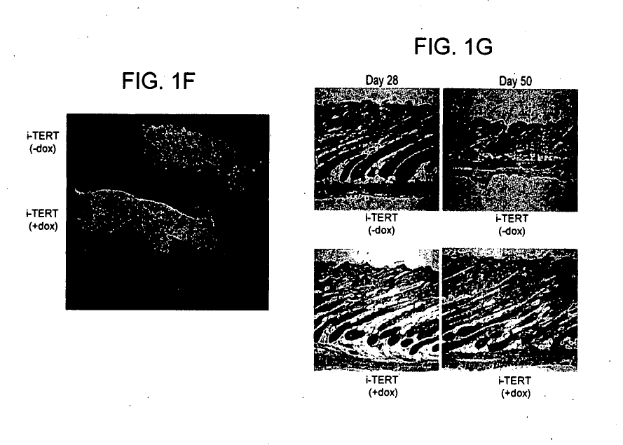

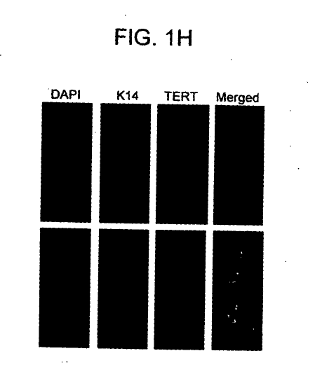

TERT was placed under control of a tetracycline-inducible promoter by subcloning a 3.5 kb EcoRI fragment of the mouse TERT cDNA into the EcoRI site of pUHD10-3. To create actin-rtTA, an EcoRI-BamHI fragment of the rtTA cDNA was subcloned into the EcoR1 site of pCAGGS by blunt-ended ligation. Prokaryotic sequences were excised from each plasmid and the gel-isolated DNA fragments were separately injected into pronuclei of FVB/N fertilized zygotes. Founder mice were screened by PCR and Southern blot. Actin-rtTA transgene positive mice were intercrossed with tetop-TERT transgene positive mice to generate actin-rtTA and tetop-TERT double transgenic mice for characterization (FIG. 1A).

TERC was placed under control of a tetracycline-inducible promoter by subcloning a 4 kb genomic fragment of the mouse TERC gene into the StuI/ApaLI site of pUHD10-3. To create actin-rtTA, an EcoRI-BamHI fragment of the rtTA cDNA was subcloned into the EcoR1 site of pCAGGS by blunt-ended ligation. Prokaryotic sequences were excised from each plasmid and the gel-isolated DNA fragments were separately injected into pronuclei of FVB/N fertilized zygotes. Founder mice were screened by PCR and Southern blot. Actin-rtTA transgene positive mice were intercrossed with tetop-TERC transgene positive mice to generate actin-rtTA and tetop-TERC double transgenic mice for characterization (FIG. 1A).

Pups derived from these crosses were genotyped by PCR using the following oligonucleotide pairs:

| | |
|---|---|
| actin-rtTA: | |
| GTGCTGGTTGTTGTGCTGTC | (SEQ ID NO.: 01) |
| GGCGAGTTTACGGGTTGT | (SEQ ID NO.: 02) |
| Tetop-TERT: | |
| GCTGGCTGCTCATTCTGTCATCTAC | (SEQ ID NO.: 03) |
| TAAAAAACCTCCCACACCTCCCCC | (SEQ ID NO.: 04) |
| Tetop-TE RC | |
| ATAAGCAGAGCTCGTTTAGTGAACC | (SEQ ID NO.: 05) |
| CCCACAGCTCAGGTAAGACA | (SEQ ID NO.: 06) |

Histology

Skin biopsies were obtained from dorsal skin of mice under anesthesia. Samples for Hematoxylin and Eosin (H&E) staining were fixed overnight in 10% formalin then embedded in paraffin. Samples for immunohistochemistry and in situ were fixed overnight in 4% paraformaldehyde followed by overnight incubation in 30% sucrose. Tissues were then embedded in OCT freezing medium and frozen on an isopropanol-dry ice slurry.

RNA In Situ Analysis

Digoxygenin-labeled anti-sense RNA probes were synthesized in vitro using digoxygenin-UTP (Roche Applied Science). In situ analysis was performed on 10 μM frozen sections or 5 μM paraffin sections. RNA in situs were developed either by indirect fluorescence using streptavidin-Cy3 (NEN Indirect Fluorescence) or by chromagenic assay using streptavidin-horse radish peroxidase and DAB (NEN Indirect Chromogenic Kit).

Immunohistochemistry

All assays were performed on 5 μM Paraffin sections. Antigen was retrieved from sections using the Vector Unmasking Kit (Vector Laboratories) according to manufacturer's instructions. Mouse monoclonal primary antibodies were detected using biotinylated anti-mouse IgG (MOM, Vector Laboratories) according to the manufacturer's protocol. Rabbit antibodies were blocked with 10% NGS diluted in TBS-T, incubated in primary antibody overnight at 4° C. and detected with FITC conjugated-anti rabbit secondary antibody (Vector Laboratories, 1:200). Primary antibodies used included mouse anti-AE13 (Sun, 1:3), mouse anti-Ki-67 (Pharmingen, 1:100), and rabbit anti-K14 (Covance, 1:500), rabbit anti-K6 (Covance, 1:500), rat-anti-CD34 (Pharmingen), and rat anti-BrdU (BD). For BrdU detection, slides were pre-treated in 1N HCL for 1 hour at 37° C.

Analysis of Label Retaining Cells

To label follicle stem cells, 10-day-old mice were injected with 250 μg of BrdU every 12 hours for four injections to mark proliferating epidermal keratinocytes. Skin samples were obtained from the mice after an extended chase period of 45-90 days. BrdU immunofluorescence was performed on frozen sections to visualize label retaining cells, followed by co-staining for CD34.

Tail Wholemount Immunolabeling

Wholemounts of tail epidermis were prepared and stained for BrdU and K14 as described in Braun et al., Development 130:5241-5255 (2003).

Northern Blots and Telomerase Activity Assays

Tissues were snap frozen in liquid nitrogen and then ground with mortar and pestle. RNA was isolated from organs or cells by means of homogenization in Trizol. 5 μg of total RNA was fractionated on a 0.8% formaldehyde gel, transferred to Hybond-N membrane, and hybridized with TERT or GAPDH ³²P-labeled DNA probes. For telomerase repeat amplification protocol (TRAP) assays, protein was extracted from 50-100 mg of tissue in CHAPS lysis buffer, and a standard TRAP reaction performed (TRAPeze).

### Example 1

### Telomerase Activity is Tightly Regulated During Mouse Postnatal Development and Hair Follicle Cycling

Telomerase is expressed in mouse stem and cancer cells and is downregulated with differentiation (Caporaso et al., 2003, Mol. Cell. Neurosci., 23:693-702; Armstrong et al., 2000, Mech. Dev., 97:109-116; Holt et al., 1996, Mol. Cell. Bio., 16:2932-2939; Allsopp et al., 2003, Blood, 102:517-520). To determine if telomerase is subject to such regulation in whole tissues, TRAP assays were performed on organs during postnatal development. During this period of development rates of proliferation diminish as morphogenesis is completed. Telomerase activity was readily detected in mouse kidney, brain, lung and skin at postnatal day 4. Enzymatic activity decreased markedly through days 10 and 21, reaching levels typical of the adult tissue by the three week timepoint.

Once down-regulated, telomerase can be reactivated in specific cellular contexts, a phenomenon well studied in lymphoid cells (Hodes et al., 2002, Nat. rev. Immunol. 2:699-706). For example, both B-cells and T-cells show elevated telomerase levels when stimulated with antigen (Weng et al., 1996, J. Exp. Med. 183:2471-2479; Ogoshi et al., 1997, J. Immunol., 158:622-628; Hathcock et al., 1998, J. Immunolo., 160:5702-5706; Weng et al., 1997, PNAS, 94:10827-10832; Hu et al., 1997, J. Immunol. 159:1068-1071). As hair follicle epithelium transitions from telogen to anagen, telomerase activity is elevated in the matrix cells of the bulb (Ramirez et al., 1997, J. Invest. Dermatol., 108:113-117). This region harbors the highly proliferative multi-potent progenitors that give rise to the cells of the hair and inner root sheath. In contrast, epithelium containing the stem cells in the bulge showed significantly lower, but measurable, levels of telomerase. These data indicate that telomerase levels increase as stem cells differentiate into progenitor cells.

To determine if telomerase is regulated similarly in hair follicles in mice, we performed TRAP assays on skin extracts. Hair follicle cycling in mice is synchronized throughout the skin for the first 60-80 days of life and the timing of these cycles has been well studied (Muller-Rover et al., 2001, J. Invest. Dermatol., 117:3-15). Skin biopsies were obtained from wild type mice and protein extracts derived from these biopsy specimens were used to program TRAP reactions. Telomerase activity in mouse skin tracked closely with the anagen phase of the hair follicle cycle (FIG. 1E). Telomerase activity was high at days 4 and 10, as follicle morphogenesis is completed during the first anagen, but decreased abruptly with regression of the follicle during catagen (day 16). Telomerase remained off during the first telogen (day 19) and was not reactivated until the second anagen (day 28). As the anagen follicle regressed, telomerase activity again declined (day 34) and remained off during the protracted resting phase of the second telogen (day 50; FIG. 1G). Therefore, the results show that telomerase activity is tightly linked to the hair follicle anagen cycle, a period of intense progenitor cell proliferation and differentiation.

### Example 2

### TERT is Conditionally Activated In Vivo in a Doxycycline-Dependent Manner

These observations reflect an association of telomerase with certain developmental states characterized by proliferation; alternatively, telomerase serves a functional role in these developmental processes independent of its function in telomere synthesis. To determine if telomerase can modulate the stem/progenitor cell program, we engineered a transgenic system in which telomerase could be conditionally activated in adult tissues using a tetracycline-inducible approach (Gossen et al., 1992, PNAS, 89:5547-5551; Furth et al., 1994, PNAS, 91:9302-9306). This conditional system is comprised of two transgenes, one in which the TERT cDNA is placed under the control of a tetracycline responsive promoter (tetop) and a second transgene which drives expression of the reverse tetracycline transactivator (rtTA). This configuration represents the tet-on approach in which the transgene is silenced until induced by treatment with the tetracycline analog, doxycycline. To drive expression of rtTA we chose a CMV enhancer/beta-actin promoter because this element was previously shown to be active in stem cells (Wright et al., 2001, Blood, 97:2278-2285) and in a broad variety of epithelial tissues (Ventela et al., 2000, Int. J. Androl., 23:236-242; Okabe et al., 1997, FEBS Lett., 407:313-319; Sawicki et al., 1998, Exp. Cell Res., 244:367-369; Akagi et al., 1997, Kidney Int., 51:1265-1269;).

Tetop-TERT+ mice were intercrossed with actin-rtTA+ mice to generate Tetop-TERT+; actin-rtTA+ (Double Tg) mice. Double Tg mice were bred off doxycycline to avoid potential adverse effects of telomerase induction on development. Based on our results showing that the adult pattern of telomerase expression is established by 21 days of age (FIG. 1A), we weaned double Tg mice and controls into cages with doxycycline-drinking water at age 21 days to characterize expression of the TERT transgene.

To assess the regulation of TERT in Double Tg mice, RNA was isolated from tissues from non-transgenic mice and from age-matched Double Tg mice treated with or without doxycycline. Northern blot analysis revealed that TERT mRNA was induced in a doxycycline-dependent manner in several tissues including skin (FIG. 1B), as well as in kidney, liver, testis, and lung. TERT mRNA was undetectable in organs from both age-matched Double Tg mice off doxycycline and from non-transgenic littermate controls. Endogenous TERT is expressed at very low levels and is not seen on Northern blots using unfractionated RNA. To determine if the induced TERT is enzymatically active, protein extracts from skin were assayed for telomerase activity by TRAP. Telomerase activity was strongly induced by doxycycline in skin from Double Tg mice, compared to Double Tg mice off doxycycline and non-transgenic controls (FIG. 1C). Therefore, the results show that both TERT mRNA and active telomerase enzyme are induced in vivo in a doxycycline-dependent manner in Double Tg mice.

### Example 3

### Induction of TERT in the Skin Alters Normal Hair Follicle Cycling

Having demonstrated that TERT mRNA is induced in a doxycycline-dependent manner, we bred additional Double Tg mice to determine the phenotypic consequences of activating TERT expression in adult mice. Double Tg mice were weaned into cages with doxycycline-drinking water at age 21 days. Within three to four weeks of doxycycline treatment, the coats of Double Tg mice were altered. The hair appeared longer and less organized than controls (FIG. 1F). In contrast, Double Tg mice off doxycycline, single Tg mice on doxycyclin and non-transgenic littermates remained unaffected. We noted that the appearance of Double Tg mice resembled that of mice with spontaneous or engineered mutations that affected hair follicle cycling (Hebert et al., 1994, Cell, 78:1017-1025; Gat et al., 1998, Cell, 95:605-614; Nakamura et al., 2001, Exp. Dermatol., 10:369-390). To investigate this phenotype further, we examined hair follicle histology after induction of TERT. Mice undergo two synchronized periods of hair follicle growth postnatally before entering a prolonged telogen phase at approximately forty days of age. To assess changes in hair follicle cycling, we analyzed skin biopsies from Double Tg mice on and off doxycycline from single transgenic mice and from non-transgenic littermates. At age 28 days, induction of TERT did not alter hair follicles; follicles in all cohorts were in anagen and these anagen follicles were histologically normal. In marked contrast, by age 50 days hair follicles from double Tg mice on doxycyline were consistently in anagen (FIG. 1G). This effect was doxycycline-dependent, occurred with 100% penetrance (18/18) and was never seen in Double Tg mice off doxycline (0/6), actin-rtTA+ single Tg mice on or off doxycycline (0/6), non-transgenic littermates (0/2) or tetop-TERT+ single Tg mice (0/3) (p=1.3×10-5 for double Tg on vs. off doxycycline by Chi square analysis) (see Table 1).

TABLE 1
Activation of TERt IN I-tert Tg Mice Promotes Anagen at Day 50

| Genotype | Doxycycline | Anagen | Telogen | Total |
|---|---|---|---|---|
| Non-Transgenic | − | 0 | 4 | 4 |
| Tetop-TERT+ | + | 0 | 4 | 4 |
| Actin-rtTA+ | + | 0 | 13 | 13 |
| i-TERT Tg | − | 0 | 9 | 9 |
| i-TERT Tg | + | 18 | 0 | 18 |

All Mice were administered doxycylcine starting day 21.
Statistical analysis was performed using chi-squared analysis.

To determine the expression pattern of transgenic TERT in skin, we performed a combination of RNA in-situ hybridization for TERT and immunohistochemistry for keratin-14, a marker of hair follicle outer root sheath. TERT mRNA was specifically detected in hair follicle epithelium and epidermis in Double Tg mice on doxycycline. TERT mRNA was detected neither in Double Tg mice off doxycycline nor in non-transgenic littermates (FIG. 1H). The distribution of TERT mRNA in Double Tg mice closely matched that of keratin-14, indicating that TERT is expressed in hair follicle epithelium upon doxycycline treatment. Together, these data show that conditional induction of TERT in hair follicle epithelium supports the anagen stage of the hair follicle cycle. Therefore, the results show that TERT causes this effect by either initiating a transition from telogen to anagen or by preventing an exit from anagen.

### Example 4

### Induction of TERC in the Skin Alters Normal Hair Follicle Cycling

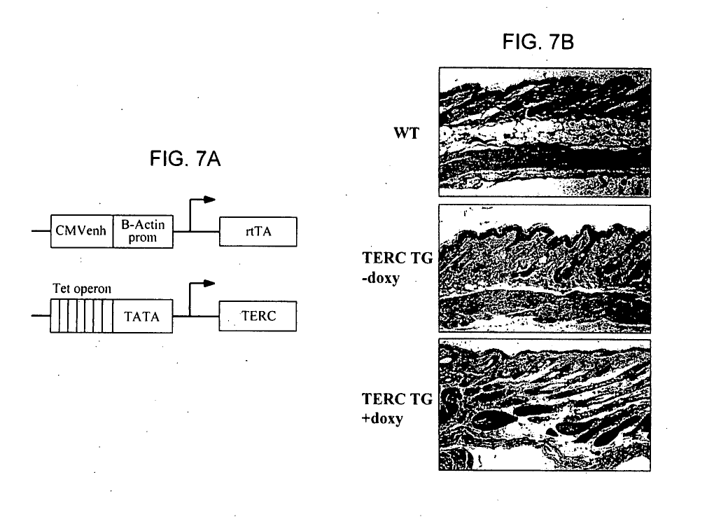

In addition to demonstrating induction of TERT in the skin alters normal hair follicle cycling, we bred additional Double Tg mice to determine the phenotypic consequences of activating TERC expression in adult mice. Double Tg mice were weaned into cages with doxycycline-drinking water at age 21 days. To investigate this phenotype changes, we examined hair follicle histology after induction of TERC. As noted above, mice undergo two synchronized periods of hair follicle growth postnatally before entering a prolonged telogen phase at approximately forty days of age. To assess changes in hair follicle cycling, we analyzed skin biopsies from Double Tg mice on and off doxycycline from single transgenic mice and from non-transgenic littermates. At age 50 days hair follicles from double Tg mice on doxycyline were consistently in anagen phase (FIG. 7B). These results demonstrate that the induction of only the TERC component of telomerase in the skin is capable of activating normal hair follicle stem cells.

### Example 5

### Induction of TERT or TERC can Initiate an Anagen Cycle and Facilitate Hair Growth

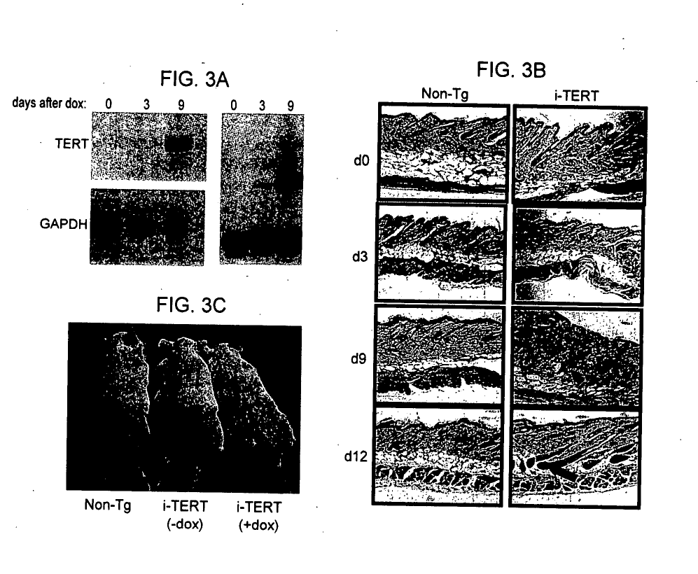

To distinguish between the possibility of TERT either initiating a transition from telogen to anagen or preventing an exit from anagen, TERT was induced in Double Tg mice after hair follicles had entered the prolonged second telogen (day 40). Double Tg mice and non-transgenic controls were treated with doxycycline beginning at day 40. Skin biopsies were obtained at regular intervals to assess the hair follicle cycle by histology, TERT expression by Northern and telomerase activity by TRAP. Histology confirmed that follicles in Double Tg and non-transgenic mice were consistently in telogen at the time of initiating doxycycline treatment (FIG. 3B). TERT mRNA was detectable by Northern blot as early as three days after initiation of doxycycline treatment in Double Tg mice (FIG. 3A). TERT levels rose incrementally from day 3 through day 9 and telomerase activity increased during this time course with similar kinetics. Histological analysis revealed that follicles in Double Tg mice remained in telogen through day 6, but by day 9 initiation of the anagen program was evident. Hair follicles had entered mid anagen of the anagen cycle (FIG. 3B) (p=0.005 by chi-squared analysis, see Table 2). By day 12, follicles in Double Tg mice were in peak anagen, as demonstrated by the presence of long follicles that penetrated the adipocyte layer and closely abutted the paniculus carnosus, the thin subcutaneous muscle layer.

TABLE 2
Activation of TERT at Day 40 in i-TERT Tg Mice Triggers
Hair Follicles to Enter Anagen by Day 50.

| Genotype | Doxycycline | Anagen | Telogen | Total |
|---|---|---|---|---|
| i-TERT Tg | − | 0 | 5 | 5 |
| i-TERT Tg | + | 3 | 0 | 3 |

Three mice were administered doxycycline at day 40, when hair follicles were in telogen. Serial biopsies were taken at time intervals after doxycycline administration.
Anagen induction occurred in all three mice by day 50.
Statistical analysis was carried out by chi squared analysis.

Hair synthesis occurs exclusively in the anagen phase during which actively proliferating matrix cells in the bulb terminally differentiate to form the keratinized cells that comprise the hair shaft. Hair growth occurs as a result of this hair formation at the follicle base that progressively pushes the protruding hair shaft further through the skin.

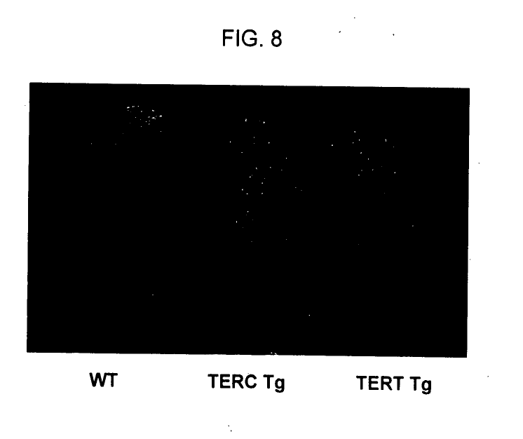

To determine if conditional activation of TERT or TERC could promote hair growth, TERT and TERC Double Tg mice were treated with doxycycline beginning in telogen (day 45). After 10 days of treatment, TERT Double Tg mice on doxycycline, TERC Double Tg mice on doxycycline, and age-matched Double Tg mice off doxycycline and as well as non-transgenic littermates were shaved dorsally. These mice were monitored for 14 days after shaving to assess rates of hair growth. Neither TERC Double Tg mice off doxycycline nor non-transgenic littermates showed significant hair growth during this interval, as anticipated because this period comprises the extended second telogen phase. In marked contrast, induction of the anagen phase of the hair cycle was associated with brisk reconstitution of the shaved hair in the Double Tg mice on doxycycline. Differences between the two groups were evident within 7 days of shaving, and by 14 days the hair in the TERC Double Tg mice on doxycycline was similar in length to unshaved mice. (FIG. 3C). In addition, by 14 days the hair in the TERT Double Tg mice on doxycycline was similar to the hair growth witnessed in the TERC Double Tg mice on doxycycline (FIG. 8). Therefore, these results show that induction of either one of TERT or TERC in hair follicles in adult mice initiates a rapid transition from the telogen phase to the anagen phase of the follicle cycle, facilitating hair growth.

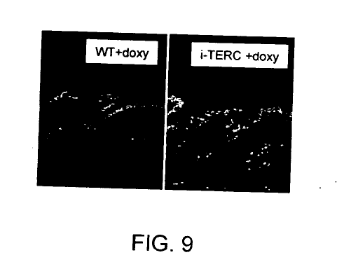

TERC activates resting stem cells and initiates a new hair growth cycle. RNA in situ hybridization was used to determine what cell types express TERC in i-TERC transgenic mice. Tissue sections from i-TERC mice on doxycycline (right panel) and wild type controls (left panel) were hybridized with an anti-sense TERC probe. As shown in FIG. 9, transgenic TERC (red) was detected in the skin epithelium, in a pattern that overlaps with keratin-14 (green), a marker of the basal layer of the epidermis and the outer root sheath of the hair follicle. This is the layer that harbors the epidermal stem cells. Induction of TERC in this layer led to a rapid transition form telogen, the resting phase of the hair follicle cycle, to anagen, the active phase. Note the longer and much deeper hair follicles in the i-TERC+ doxycycline samples. All controls including wild type mice and i-TERC mice off doxycycline remained in telogen during these experiments. To determine if activation of TERC could encourage hair growth, mice were shaved during the second postnatal telogen period and rates of hair growth were assessed. Induction of TERC in i-TERC mice caused rapid hair growth. In contrast, control mice showed no hair growth during this period. These effects were similar to those of TERT. Therefore, the results show that expression of TERC activates quiescent stem cells, causes a rapid transition from telogen to anagen and facilitates hair growth. The effects of TERC on hair growth are similar to those of TERT as described above.

### Example 6

### Induction of TERT does not Interfere with Normal Cell Differentiation in the Hair Follicle

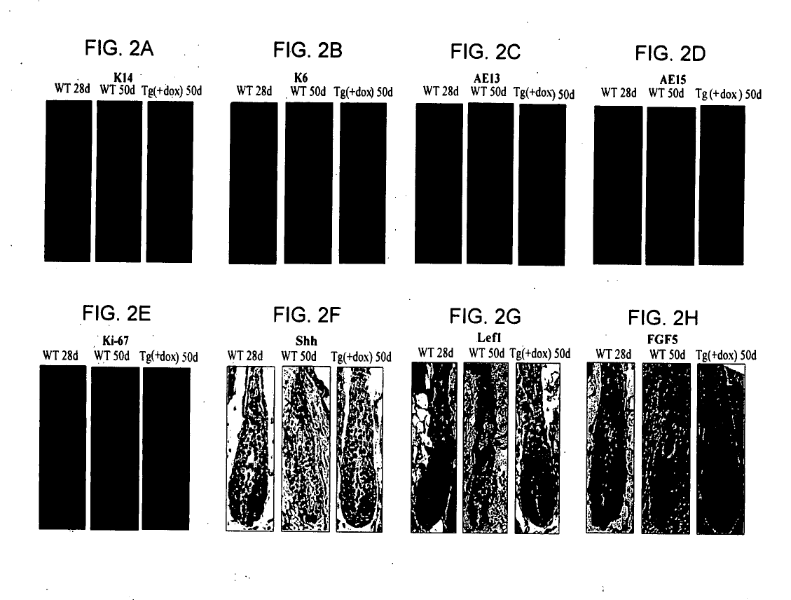

Hair follicle cycling is a complex signaling program involving self-renewal, proliferation, multilineage differentiation, and apoptotic regression. Many of the classical pathways that control hair follicle morphogenesis and cycling also contribute to proper differentiation of hair follicle keratinocytes. For example, activation of the Wnt/β-catenin pathway can lead to induction of anagen, but alters differentiation of the inner root sheath. Prolonged activation can also lead to sever hyperplastic hair follicles and de novo hair follicle formation (Gat et al., 1998; Van Mater et al., 2003, Genes Dev., 17:1219-1224). In fact, separating the timing of hair follicle cycling from cell differentiation has been difficult. To determine if persistent TERT expression altered differentiation in TERT-induced anagen follicles, we assessed keratinocyte differentiation in these follicles through the use of well-established markers that identify specific cellular compartments of the hair follicle. TERT-induced anagen follicles in 50 day old Double Tg mice were compared to the second postnatal, anagen in non-transgenic mice (day 28) and age-matched 50 day old non-transgenic mice in telogen. The pattern of expression of keratin-14 was identical in TERT-induced anagen follicles and non-transgenic anagen hair follicles, indicating normal differentiation of the outer root sheath (FIG. 2A). Similarly, expression patterns for keratin-6 (inner layer of the outer root sheath), AE-13 (hair keratins), and AE-15 (outer root sheath) were identical in both TERT-induced anagen and normal non-transgenic anagen follicles (FIGS. 2B, 2C and 2D). The dermal papilla was detected by alkaline phosphatase staining and was shown to have a location and structure similar in TERT-induced anagen and non-transgenic anagen follicles. Finally, cell proliferation in TERT-induced anagen follicles was assessed using the Ki-67 marker that identifies cells in active phases of the cell cycle (FIG. 2E). The transit amplifying matrix cells comprised the majority of Ki-67+ cells in both normal anagen and TERT-induced anagen follicles. Despite expression of transgenic TERT mRNA throughout the epithelium of the follicle, active proliferation was restricted to the progenitor cell population in the bulb region. The absence of aberrant differentiation or aberrant proliferation in TERT-induced anagen follicles shows that TERT acts in this setting by altering the timing of hair follicle cycling.

### Example 7

### TERT's Effect is Mediated by the Stem Cell Compartment

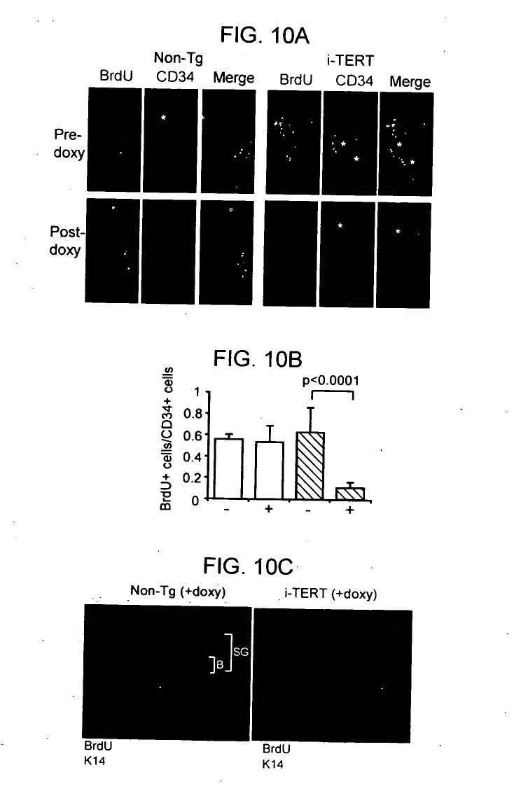

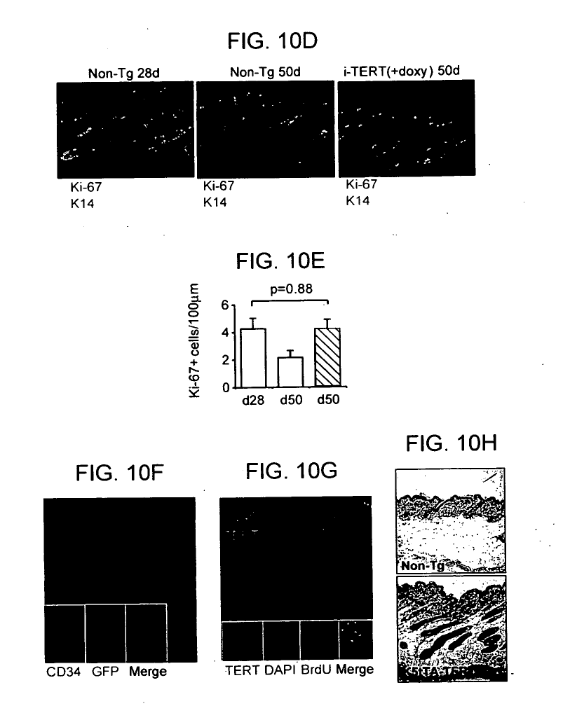

Because activation of bulge stem cells is integral to the initiation of a new anagen cycle (Cotsarelis, et al. Cell 61, 1329-37 (1990); Taylor, et al. Cell 102, 451-61 (2000); and Tumbar, et al. Science 303, 359-63 (2004)), it was hypothesized that TERT's effects on the hair follicle cycle might be mediated through the stem cell compartment. To address this hypothesis, a label retaining technique was employed that has been Used successfully to mark hair follicle bulge stem cells by repeated injections of BrdU followed by a long chase period (Cotsarelis, et al. Cell 61, 1329-37 (1990)). Cohorts of i-TERT mice and non-transgenic controls were injected with BrdU at 10 days of age. During the second telogen, mice in each group were biopsied, switched to doxycycline drinking water, and biopsied again between days 80 and 100. Label retaining cells (LRCs) were visualized by double immunostaining with antibodies against BrdU and CD34 (Trempus, et al. J Invest Dermatol 120, 501-11 (2003); Blanpain, et al. Cell 118, 635-48 (2004)). LRCs were present in similar numbers in both i-TERT and non-transgenic mice at age 55 days, before the switch to doxycycline water (approximately 0.6 BrdU+ cells/CD34+ cell). After five weeks of doxycycline treatment, BrdU label in CD34+ stem cells was retained in non-transgenic mice at comparable levels, consistent with previous observations that BrdU label persists in quiescent bulge cells for more than six months. In marked contrast, BrdU label was profoundly depleted in the CD34+ cell population in the bulge by induction of TERT in i-TERT mice (76% reduction in BrdU+ cells/CD34+ cell, p<0.0001) (FIGS. 9A and 9B). Despite the loss of BrdU label, CD34+ cells in the bulge remained in similar numbers, indicating that, under the influence of TERT, stem cells divide but likely self-renew to maintain the CD34+ population. A similar reduction in LRCs in i-TERT mice was seen in epidermal wholemounts, corroborating the effects seen in dorsal skin sections (FIG. 10C). These results show that TERT causes hair follicle bulge cells to proliferate, diluting BrdU label from this quiescent stem cell population.

To determine if TERT more broadly enhances keratinocyte proliferation, the proliferation index in the basal layer of the interfollicular epidermis was measured (FIG. 10D). Despite expression of transgenic TERT mRNA in this compartment, proliferation was not substantially altered in the basal layer in i-TERT mice compared to non-transgenic littermates in anagen (4.2 Ki-67+ cells/100 μm for i-TERT day 50 compared to 4.3 Ki-67+ cells/100 μm for non-transgenic day 28) (FIG. 10E). Furthermore, no changes in structure, differentiation, or signaling in either hair follicle or interfollicular epidermis in i-TERT mice were observed. Therefore, the results show that the principle effects of TERT in this system occur through activation of quiescent hair follicle stem cells.

To determine if these results are consistent with a direct effect of TERT on stem cells, the expression of transgenic TERT in the stem cell compartment was assessed. It was found that the promoter element used to direct rtTA expression is strongly active in CD34+ bulge cells in actin-GFP mice (FIG. 10F). Furthermore, TERT mRNA was co-expressed with BrdU in LRCs in the bulge region in i-TERT mice (FIG. 10G). While induction of anagen can occur through signals from the dermal papilla (Sato, et al. J Clin Invest 104, 855-64 (1999)), the lack of detectable levels of TERT mRNA in the dermal papilla makes it unlikely in this case. To confirm that TERT exerts its effect through the epithelium, tetop-TERT mice were intercrossed with a transgenic mouse in which the Keratin-5 promoter drives expression of the tetracycline transactivator (tTA) in the basal layer and outer root sheath (K5-tTA, tet off configuration). Compound K5-tTA⁺; tetop-TERT⁺ mice were bred on doxycycline and weaned off doxycycline-drinking water at day 21 to induce the TERT transgene. Expression of TERT mRNA in skin epithelium (data not shown) induced anagen in 5/5 K5-tTA⁺; tetop-TERT⁺ mice, whereas all littermate control biopsies were in telogen (6/6, p=0.0009 by Chi square analysis)(FIG. 10H). These results show that TERT's effects in promoting anagen are intrinsic to the K5 compartment of the skin epithelium, the layer where the hair follicle stem cells reside.

### Example 8

### Classical Anagen Signaling Pathways are Active in the TERT-Induced Anagen Follicles

Although the signals that govern induction of anagen are incompletely understood, reciprocal inductive interactions between the dermal papilla and epithelium play a critical role. Secreted morphogens, including Wnts, Shh, and noggin proteins, contribute to both follicle morphogeneis and follicle cycling. Because TERT induced a transition from telogen to anagen, we wondered if conditional TERT activation could substitute for any of these pathways. Shh is required for hair follicle morphogenesis and is expressed only during anagen at the base of the hair follicle (Oro et al., 2003, Dev. Biol., 255:238-248). Shh was expressed appropriately by RNA in-situ in an asymmetrical distribution of TERT-induced anagen follicles (FIG. 2F). Wnt/β-catenin signaling is also critical for follicle morphogenesis and follicle cycling. Loss of β-catenin or its partner, the transcription factor LEF-1, impairs follicle development (Huelsken et al., 2001, Cell, 105:533-545). In contrast overexpression of β-catenin can induce anagen and de novo follicle morphogenesis (Gat et al., 1998; Van Meter et al., 2003; Van Genderen et al., Genes Dev., 8:2691-2703). We therefore assessed the integrity of the β-catenin pathway by assaying expression of LEF-1. Lef-1 was expressed in the bulb region of TERT-induced anagen follicles and this pattern was indistinguishable from its distribution in normal anagen (FIG. 2G).

Finally, we examined two inhibitory pathways, FGF5 and BMP4. FGF5 is a secreted protein expressed in the outer root sheath during the anagen VI phase of the hair growth cycle. Studies have shown that FGF5 functions as an inhibitor of hair elongation by contributing to the signal that instructs follicles to exit anagen (Hebert et al., 1994: Sundberg et al., 1997, Vet. Pathol., 34:171-179). BMP4 has been implicated in inhibiting the induction of many ectodermal derivatives and is thought to be an inhibitor of anagen initiation and progression in postnatal skin by antagonizing the positive effects of noggin (Oro et al., 1998, Cell, 95:575-578). We considered the possibility that TERT could down regulate normal inhibitory signals and thus lead to hair follicles being trapped in anagen. However, expression levels and patterns of both BMP4 and FGF5 in TERT-induced anagen were similar to those in normal anagen (FIG. 2H). Together these data show that conditional activation of TERT can initiate the anagen program, but that this program unfolds under the influence of a similar set of morphogens including Wnt and Shh.

### Example 9

### Conditional TERT Activation does not Cause Telomere Uncapping

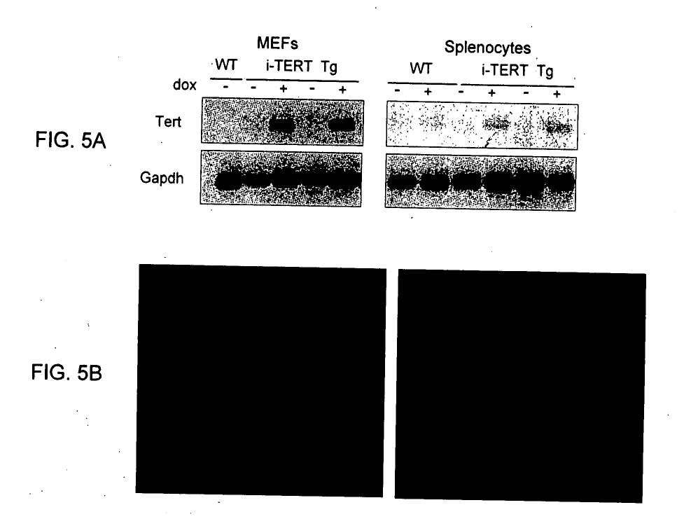

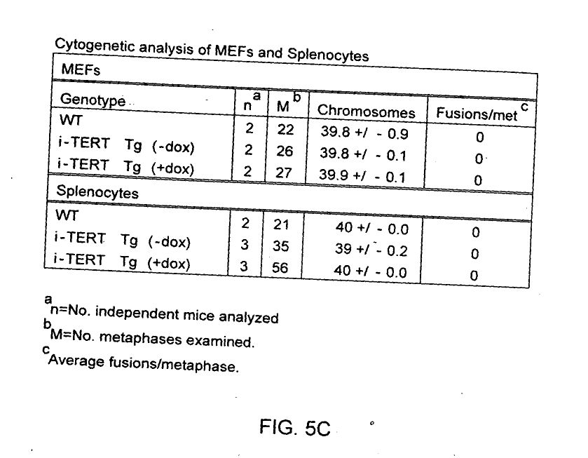

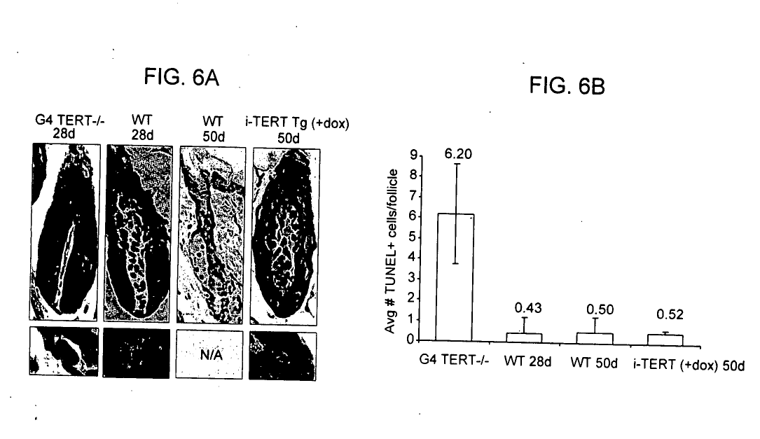

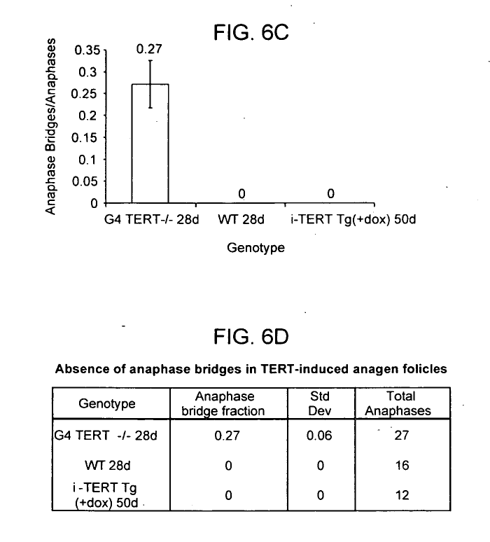

Telomere uncapping can occur as telomeres progressively shorten and the shortest telomeres can no longer support the protected structure at the chromosome end. Long telomeres are also subject to uncapping, in the context of overexpression of some telomere binding proteins and telomerase components. Est1A and dominant-negative TRF2 each lead to rapid telomere uncapping when expressed in human cells (Reichenbach et al., 2003, Curr. Biol., 13:568-574; Smogorzewska et al., EMBO J., 21:4338-4348). In contrast, expression of TERT in culture results in telomere synthesis and immortalization (Counter et al., 1992, EMBO J., 11:1921-1929). Nonetheless, we wished to rule out an unanticipated effect of TERT on telomere stability. The hallmark of telomere uncapping is chromosomal end-to-end fusion (Mathieu et al., 2004, Cell. Mol. Life Sci., 61:641-656). To determine how conditional TERT activation affects telomere function, we derived mouse embryo fibroblasts and splenocytes from Double Tg mice and non-transgenic controls. TERT mRNA was induced in a doxycycline-dependent manner in both MEFs and splenocyte cultures (FIG. 5A). Analysis of metaphase preparations from MEFs and splenocytes showed no increase in chromosomal end-to-end fusions with TERT induction (FIG. 5B). To determine if TERT induction caused telomere uncapping in the epithelium of the hair follicle, we measured rates of apoptosis in anagen follicles form Double Tg mice and non-transgenic controls. Telomere dysfunction in late generation telomerase-deficient mice causes significantly elevated rates of apoptosis in regenerating tissues (Wong et al., 2003, Nature, 421:643-648; Hemann et al., Mol. Biol. Cell, 12:2023-2030). Apoptosis was therefore measured by TUNEL assay on TERT-induced anagen follicles and on non-transgenic anagen follicles. The frequency of apoptotic nuclei in both groups was less than 0.5 per follicle. In contrast, anagen follicles from late generation TERT−/− mice showed a rate of 8 apoptotic nuclei per follicle (FIG. 5A and FIG. 5B). Mitotic figures are abundant in anagen follicles because of the high rates of cell division in the matrix cell population. Fused chromosomes result in anaphase bridges during mitosis as dicentric chromosomes are pulled to opposite spindle poles. We measured rates of anaphase bridge formation in anagen follicles as an independent measure of telomere dysfunction in this compartment. Anagen follicles from late generation TERT−/− mice showed frequent anaphase bridges. In contrast, anaphase bridges were seen neither in TERT-induced anagen follicles nor in non-transgenic anagen follicles (FIGS. 5A, 5C, and 5D). Therefore, the results show that conditional activation of TERT does not result in telomere uncapping as measured by cytogenetics, rates of apoptosis, and frequency of anaphase bridge formation.

### Example 10

### TERT Induces Anagen Through a Telomerase RNA Component-Independent Mechanism

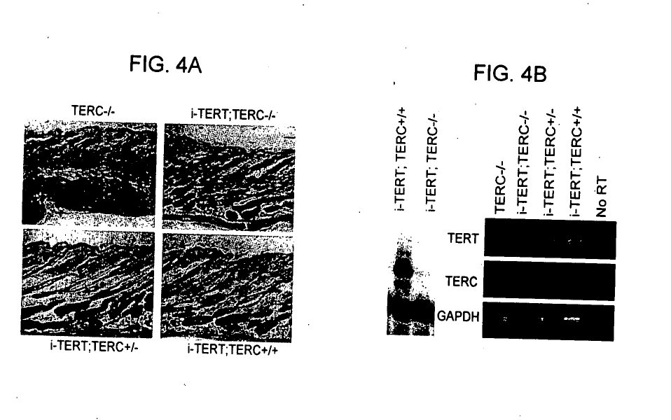

We hypothesized that the effects of TERT on the hair follicle could occur through one of two mechanisms. TERT activation could extend telomeres through de novo nucleotide addition to the telomere end. Enzymatic action at the telomere, or increased telomere length itself, could result in a signal that led to activation of the anagen program. Alternatively, the TERT protein may signal hair follicle activation independent of its role in telomere synthesis. One way of distinguishing these two models genetically is to determine if the function of TERT in the hair follicle requires the telomerase RNA component telomere synthesis requires both TERT and TERC, therefore if the effects of TERT are retained in a TERC−/− background, telomere extension cannot be required. Toward this end, TERC+/− mice in a mixed genetic background were backcrossed to FVB/N for six generations. Once on a pure background, TERC+/− mice were intercrossed with inducible TERT alleles to derive cohorts of Double Tg mice that are TERC+/+, TERC+/− and TERC−/−. Mice in each group were treated with doxycycline at age 21 days and monitored phentoypically and through skin biopsies. After four weeks on doxycycline, Double Tg T TERC−/− mice showed longer, disorganized hair and were indistinguishable from their Double Tg TERC+/+ or Double Tg TERC+/− littermates. Histological analysis at day 50 revealed that conditional activation of TERT induced anagen even in 5/5 Double Tg TERC−/− mice (FIG. 4A) (p=0.0003 for i-TERT Tg; TERC−/− plus doxycycline vs. i-TERT Tg mice off doxyxyxline (0/8 in anagen), see Table 3). These results were identical to Double Tg mice in either TERC+/+ or TERC+/− backgrounds (6/6 in anagen), showing that TERC is not required for TERT to induce anagen. Skin biopsies from i-TERT Tg TERC−/− mice were shown to lack TRAP activity and TERC RNA (FIG. 4B). These data prove that the effects of TERT in activating hair follicles to transition from telogen to anagen are independent of TERT's function in telomere synthesis.

TABLE 3
TERT Can Induce an Anagen Phase in the Absence of TERC

| Genotype | Doxycycline | Anagen | Telogen | Total |
|---|---|---|---|---|
| i-TERT Tg Controls | − | 0 | 8 | 8 |
| i-TERT Tg; TERC+/− | + | 5 | 0 | 5 |
| i-TERT Tg; TERC−/− | + | 5 | 0 | 5 |

Number of mice that were analyzed in TERC−/−, TERC+/−, or TERC+/+ backgrounds.
Mice were administered doxycycline starting day 21 and was biopsied at day 50.
Each biopsy was categorized as anagen or telogen based on histology.
Statistical analysis was performed using chi squared analysis against controls.

These observations define a new set of TERT activities that do not involve enzymatic action at the telomere end. The results in the hair follicle show that these non-canonical functions of TERT serve to activate resting stem cells in the follicle bulge region. TERT induction in this setting promotes the transition from resting stem cell to actively proliferating progenitor cell. In this way, TERT initiates the anagen program in the follicle. The data also show that once initiated, the follicle relies on the same set of intricate signaling programs that guide hair follicle development, including Shh and Wnt/β-catenin. Therefore, the results show that TERT is capable of initiating a program of organ development that relies upon a complex set of morphogens for its accurate completion.

It is evident from the above results and discussion that the subject invention provides for highly efficient methods and compositions for activating a cell, which can be employed in the treatment of disorders in which it is beneficial to progress a target cell from a first quiescent state to a second non-quiescent state. As such, the present invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method for activating a cell comprising:
conditionally increasing transcription of a coding sequence of only one of:
(a) a telomerase reverse transcriptase (TERT), or
(b) a telomerase RNA component (TERC);
in said cell in a manner sufficient to activate said cell.

2. The method according to claim 1, wherein said method conditionally increases transcription of a TERT coding sequence.

3. The method according to claim 1, wherein said method conditionally increases transcription of a TERC coding sequence.

4. The method according to claim 1 wherein said method comprises introducing into said cell an agent that conditionally increases transcription of said coding sequence.

5. The method according to claim 4, wherein said agent activates a conditional promoter system operably linked to said coding sequence.

6. The method according to claim 1, wherein said method comprises introducing into said cell a nucleic acid vector comprising an expression system having a conditional promoter system operably linked to said coding sequence.

7. The method according to claim 6, wherein said conditional promoter system comprises a tetracycline inducible promoter.

8. The method according to claim 1, wherein said method is in vitro.

9. The method according to claim 1, wherein said method is in vivo.

10. The method according to claim 8, wherein said cell is present in a mammal.

11. The method according to claim 9, wherein said mammal is a human.

12. The method according to claim 1, wherein said cell is a hair follicle cell.

13. The method according to claim 1, wherein said cell is a pancreatic islet cell.

14. The method according to claim 1, wherein said cell is a neuronal cell.

15. The method according to claim 1, wherein said cell is a bone marrow cell.

16. A method for activating a cell in a host comprising:
administering to said host an effective amount of an agent that conditionally increases transcription of a coding sequence of only one of:
(a) a telomerase reverse transcriptase (TERT), or
(b) a telomerase RNA component (TERC);
to activate said cell.

17. The method according to claim 16, wherein said method conditionally increases transcription of a TERT coding sequence.

18. The method according to claim 16, wherein said method conditionally increases transcription of a TERC coding sequence.

19. The method according to claim 16, wherein said agent activates a conditional promoter system operably linked to said coding sequence.

20. The method according to claim 19, wherein said conditional promoter system comprises a tetracycline inducible promoter.

21. The method according to claim 16, wherein said host is a mammal.

22. The method according to claim 21, wherein said mammal is a human.

23. The method according to claim 16, wherein said cell is a hair follicle cell.

24. The method according to claim 16, wherein said cell is a pancreatic islet cell.

25. The method according to claim 16, wherein said cell is a neuronal cell.

26. The method according to claim 16, wherein said cell is a bone marrow cell.

27. A method for activating a hair follicle cell in a host in vivo comprising:
administering to said host an effective amount of an agent that conditionally increases transcription of a coding sequence of only one of:
(a) a telomerase reverse transcriptase (TERT); or
(b) a telomerase RNA component (TERC);
to activate said hair follicle cell.

28. The method according to claim 27, wherein said method conditionally increases transcription of a TERT coding sequence.

29. The method according to claim 27, wherein said method conditionally increases transcription of a TERC coding sequence.

30. The method according to claim 27, wherein said agent activates a conditional promoter system operably linked to said coding sequence.

31. The method according to claim 30, wherein said conditional promoter system comprises a tetracycline inducible promoter.

32. The method according to claim 27, wherein said host is a mammal.

33. The method according to claim 32, wherein said mammal is a human.

34. The method according to 27, wherein said activation of said hair follicle cells results in hair growth.

35. A transgenic animal, wherein said transgenic animal conditionally transcribes only one of:
(a) a telomerase reverse transcriptase (TERT) transgene; or
(b) a telomerase RNA component (TERC) transgene.

36. The transgenic animal according to claim 35, wherein said transgenic animal comprises a TERT transgene.

37. The transgenic animal according to claim 35, wherein said transgenic animal comprises a TERC transgene.

38. The transgenic animal according to claim 35, wherein said animal is a mammal.

39. The transgenic animal according to claim 38, wherein said mammal is a rodent.

40. The transgenic animal according to claim 38, wherein said conditional transcription is provided by a conditional promoter system operably linked to said TERT transgene or TERC transgene.

41. The transgenic animal according to claim 40, wherein said conditional promoter system is a tetracycline inducible promoter system.

42. A method for identifying a compound that is capable of modulating the activity of one of a telomerase reverse transcriptase (TERT) or a telomerase RNA component (TERC), said method comprising:
(a) activating a cell by conditionally increasing transcription of a coding sequence of only one of:
(i) said TERT; or
(ii) said TERC;
(b) contacting said compound to said cell; and
(c) observing the effect of said compound on said cell.

43. The method according to claim 42, wherein said activating comprises conditionally increasing transcription of a TERT coding sequence.

44. The method according to claim 42, wherein said activating comprises conditionally increasing transcription of a TERC coding sequence.

45. The method according to claim 42, wherein said activating comprises administering to said cell an agent that conditionally increases transcription of said coding sequence.

46. The method according to claim 45, wherein said activating comprises administering an agent that activates a conditional promoter system operably linked to said coding sequence.

47. The method according to claim 42, wherein said method further comprises introducing into said cell a nucleic acid vector comprising an expression system having a conditional promoter system operably linked to said coding sequence.

48. The method according to claim 47, wherein said conditional promoter system comprises a tetracycline inducible promoter.

49. The method according to claim 42, wherein said cell is in a mammal.

50. The method according to claim 49, wherein said mammal is a rodent.

51. The method according to claim 42, wherein said compound is a polypeptide.

52. The method according to claim 42, wherein said compound is a nucleic acid.

53. The method according to claim 42, wherein said compound is small molecule.

54. The method according to claim 42, wherein said modulating is enhancing activity.

55. The method according to claim 42, wherein said modulating is repressing activity.

56. A system for use in identifying a compound that is capable of modulating the activation of either a telomerase reverse transcriptase (TERT) or a telomerase RNA component (TERC) said method comprising;
(a) a transgenic animal conditionally transcribing only one of:
(i) said TERT transgene, or
(ii) said TERC transgene; and
(b) an agent that activates conditional transcription of said transgene.

57. The system according to claim 56, wherein said conditional transcription is provided by a conditional promoter system operably linked to said TERT transgene or TERC transgene.

58. The system according to claim 57, wherein said conditional promoter system is the tetracycline inducible promoter system.

59. The system according to claim 56, wherein said animal is a mammal.

60. The system according to claim 56, wherein said mammal is a mouse.

61. The system according to claim 56, wherein said agent is doxycycline or an analog thereof.

62. A conditional expression vector, comprising
a conditional promoter system operably linked to the coding sequence of only one of:
(a) a telomerase reverse transcriptase (TERT), or
(b) a telomerase RNA component (TERC).

63. The conditional expression vector according to claim 62, wherein said conditional promoter system is a tetracycline inducible promoter system.

64. A system for use in producing a conditional expression animal model comprising:
(a) a conditional expression vector comprising a conditional promoter system operably linked to the coding sequence of only one of:
(a) a telomerase reverse transcriptase (TERT), or
(b) a telomerase RNA component (TERC); and
(b) an animal.

65. The system according to claim 64, wherein said conditional promoter system is a tetracycline inducible promoter system.

66. The system according to claim 64, wherein said animal is a mammal.

67. The system according to claim 66, wherein said mammal is a rodent.


SEQUENCE LISTING (6 sequences)

SEQ ID NO: 1 - DNA, 20 bp, Artificial Sequence
  Primer
gtgctggttgttgtgctgtc

SEQ ID NO: 2 - DNA, 18 bp, Artificial Sequence
  Primer
ggcgagtttacgggttgt

SEQ ID NO: 3 - DNA, 25 bp, Artificial Sequence
  Primer
gctggctgctcattctgtcatctac

SEQ ID NO: 4 - DNA, 24 bp, Artificial Sequence
  Primer
taaaaaacctcccacacctccccc

SEQ ID NO: 5 - DNA, 25 bp, Artificial Sequence
  Primer
ataagcagagctcgtttagtgaacc

SEQ ID NO: 6 - DNA, 20 bp, Artificial Sequence
  Primer
cccacagctcaggtaagaca